(19) United States
(12) Patent Application Publication
(10) Pub. No.: US 2023/0355617 A1
(43) Pub. Date: Nov. 9, 2023

(54) SMALL MOLECULE INHIBITORS OF LEMUR TYROSINE KINASE 3

(71) Applicant: The University of Sussex, Brighton (GB)

(72) Inventors: George GIAMAS, Brighton (GB); John SPENCER, Brighton (GB)

(73) Assignee: The University of Sussex, Brighton, GB (GB)

(21) Appl. No.: 18/026,054

(22) PCT Filed: Sep. 14, 2021
(86) PCT No.: PCT/GB2021/052382

§ 371 Date: Aug. 25, 2023

(30) Foreign Application Priority Data
Sep. 14, 2020 (GB) 2014407.7
Sep. 21, 2020 (GB) 2014873.0

(51) Int. Cl. A61K 31/5025; A61K 31/433; A61P 35/00

(52) U.S. Cl. CPC A61K 31/5025; A61K 31/433; A61P 35/00

(57) ABSTRACT

The present invention relates to compounds of formula (I) and compositions comprising the same. The compounds and compositions may be used treat, prevent or ameliorate diseases treatable by inhibition of the Lemur tyrosine kinase 3 (LMTK3), such as cancer.

The invention relates to cancer, and in particular to novel compositions and therapies for use in treating, preventing or ameliorating diseases treatable by inhibition of the Lemur tyrosine kinase 3 (LMTK3), such as cancer.

Protein kinases play a pivotal role in regulating intracellular signal transduction pathways involving almost every aspect of cell activity including proliferation, survival, differentiation, apoptosis, metabolism, angiogenesis, immune surveillance and motility. Perturbation of their signaling affects their activities, which contributes to human diseases including malignancies. Targeted therapies against kinases have improved the clinical outcome of patients in the past decade. However, resistance to these treatments often develops, largely due to the aberrant activation of other kinases possessing a complementary or compensatory function and the inventors have shown redundancy in kinase signaling in broad networks (Stebbing J, et al. (2015) Characterization of the Tyrosine Kinase-Regulated Proteome in Breast Cancer by Combined use of RNA interference (RNAi) and Stable Isotope Labeling with Amino Acids in Cell Culture (SILAC) Quantitative Proteomics. Mol Cell Proteomics 14(9)2479-2492).

The oncogenic role of Lemur tyrosine kinase 3 (LMTK3) has been established over the last years, supported by mechanistic and translational data in different tumor types and settings. Since LMTK3 has been proposed as a potential new therapeutic target in breast cancer and considering its involvement in other tumors, there is a pressing need to further decipher the signaling pathways in which LMTK3 is implicated in and identify potent, selective, cell permeable small molecule inhibitors that can be used to enable pathway investigation and in doing so also establish onward tractability for future translational activities.

The present invention arises from the inventors work in attempting to identify inhibitors of LMTK3.

In accordance with a first aspect of the invention, there is provided a compound of formula (I):

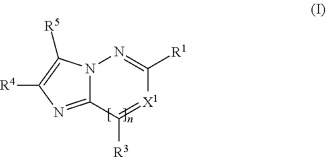

wherein R¹ is an optionally substituted C₆-C₁₂ aryl, an optionally substituted 5 to 10 membered heteroaryl, an optionally substituted 3 to 10 membered heterocycyl, L¹L²R⁸ or a halogen, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;

n is o and X¹ is S, O or NR²; or n is 1 and X¹ is CR² or N;

R² to R⁴ are independently hydrogen, a halogen, an optionally substituted C₁-C₁₅ alkyl, an optionally substituted C₂-C₁₅ alkenyl or an optionally substituted C₂-C₁₅ alkynyl; R⁵ is an optionally substituted C₆-C₁₂ aryl, an optionally substituted 5 to 10 membered heteroaryl, an optionally substituted 3 to 10 membered heterocycyl, or L¹L²R⁸, wherein the aryl, heteroaryl or heterocycyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, OR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶; and

R⁶ and R⁷ are independently H, optionally substituted C₁-C₁₅ alkyl, optionally substituted C₂-C₁₅ alkenyl or optionally substituted C₂-C₁₅ alkynyl;

R⁸ is OR⁶, SR⁶, NR⁶R⁷, CONR⁶R⁷, CN, COOR⁶, NO₂, NR⁶COR⁷, NR⁶SO₂R⁷, OC(O)OR⁶, OC(O)NR⁶R⁷, OC(O)R⁶, an optionally substituted C₆-C₁₂ aryl, an optionally substituted 5 to 10 membered heteroaryl, an optionally substituted 3 to 10 membered heterocycyl, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;

L¹ is absent or is O, S or NR⁶; and

L² is absent or is an optionally substituted C₁ to C₁₅ alkylene or an optionally substituted C₂ to C₁₅ alkylyne;

or a pharmaceutically acceptable complex, salt, solvate, tautomeric form or polymorphic form thereof for use in therapy.

The inventors have found that compounds of formula (I) can inhibit LMTK3, and may be used to treat cancer.

In accordance with a second aspect, there is provided a compound of formula (I):

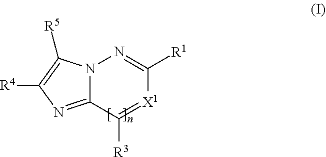

wherein R¹ is an optionally substituted C₆-C₁₂ aryl, an optionally substituted 5 to 10 membered heteroaryl, an optionally substituted 3 to 10 membered heterocycyl, L¹L²R⁸ or a halogen, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;

n is o and X¹ is S, O or NR²; or n is 1 and X¹ is CR² or N;

R² to R⁴ are independently hydrogen, a halogen, an optionally substituted C₁-C₁₅ alkyl, an optionally substituted C₂-C₁₅ alkenyl or an optionally substituted C₂-C₁₅ alkynyl;

R⁵ is an optionally substituted C₆-C₁₂ aryl, an optionally substituted 5 to 10 membered heteroaryl, an optionally substituted 3 to 10 membered heterocycyl, or L¹L²R⁸, wherein the aryl, heteroaryl or heterocycyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, OR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶; and

R⁶ and R⁷ are independently H, optionally substituted C₁-C₁₅ alkyl, optionally substituted C₂-C₁₅ alkenyl or optionally substituted C₂-C₁₅ alkynyl;

R⁸ is OR⁶, SR⁶, NR⁶R⁷, CONR⁶R⁷, CN, COOR⁶, NO₂, NR⁶COR⁷, NR⁶SO₂R⁷, OC(O)OR⁶, OC(O)NR⁶R⁷, OC(O)R⁶, an optionally substituted C₆-C₁₂ aryl, an optionally substituted 5 to 10 membered heteroaryl, an optionally substituted 3 to 10 membered heterocycyl, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;

L¹ is absent or is O, S or NR⁶; and

L² is absent or is an optionally substituted C₁ to C₁₅ alkylene or an optionally substituted C₂ to C₁₅ alkylyne;

or a pharmaceutically acceptable complex, salt, solvate, tautomeric form or polymorphic form thereof for use in treating a disease treatable by inhibiting Lemur tyrosine kinase 3 (LMTK3).

In a third aspect, there is provided a method of treating, preventing or ameliorating a disease treatable by inhibiting Lemur tyrosine kinase 3 (LMTK3) in a subject, the method comprising administering to a subject in need of such treatment, a therapeutically effective amount of a compound of formula (I):

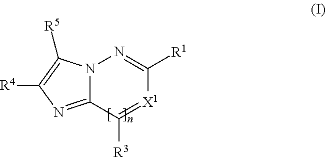

wherein R¹ is an optionally substituted C₆-C₁₂ aryl, an optionally substituted 5 to 10 membered heteroaryl, an optionally substituted 3 to 10 membered heterocycyl, L¹L²R⁸ or a halogen, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;

n is o and X¹ is S, O or NR²; or n is 1 and X¹ is CR² or N;

R² to R⁴ are independently hydrogen, a halogen, an optionally substituted C₁-C₁₅ alkyl, an optionally substituted C₂-C₁₅ alkenyl or an optionally substituted C₂-C₁₅ alkynyl;

R⁵ is an optionally substituted C₆-C₁₂ aryl, an optionally substituted 5 to 10 membered heteroaryl, an optionally substituted 3 to 10 membered heterocycyl, or L¹L²R⁸, wherein the aryl, heteroaryl or heterocycyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, OR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶; and

R⁶ and R⁷ are independently H, optionally substituted C₁-C₁₅ alkyl, optionally substituted C₂-C₁₅ alkenyl or optionally substituted C₂-C₁₅ alkynyl;

R⁸ is OR⁶, SR⁶, NR⁶R⁷, CONR⁶R⁷, CN, COOR⁶, NO₂, NR⁶COR⁷, NR⁶SO₂R⁷, OC(O)OR⁶, OC(O)NR⁶R⁷, OC(O)R⁶, an optionally substituted C₆-C₁₂ aryl, an optionally substituted 5 to 10 membered heteroaryl, an optionally substituted 3 to 10 membered heterocycyl, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;

L¹ is absent or is O, S or NR⁶; and

L² is absent or is an optionally substituted C₁ to C₁₅ alkylene or an optionally substituted C₂ to C₁₅ alkylyne;

or a pharmaceutically acceptable complex, salt, solvate, tautomeric form or polymorphic form thereof.

Preventing may be understood to mean reducing the likelihood of.

The disease may be cancer, attention deficit hyperactivity disorder (ADHD), hyper-sociability, a prepulse inhibition (PPI) deficit, cognitive dysfunction or a neurodegenerative disease. The neurodegenerative disease may be Alzheimer's or Parkinson's disease.

In a preferred embodiment, the disease is cancer. The cancer may be blood cancer, brain cancer, breast cancer, cervical cancer, colon cancer, endometrial cancer, gastric cancer, leukemia, liver cancer, lung cancer, a lymphoma, ovarian cancer, pancreatic cancer, prostate cancer, renal cancer or skin cancer. The lung cancer may be non-small-cell lung carcinoma (NSCLC). The lymphoma may be a central nervous system (CNS) lymphoma. The skin cancer may be melanoma.

Preferably, the cancer is breast cancer. More preferably, the cancer is breast cancer. The breast cancer may be a triple-negative-breast cancer (e.g. ER−/PR−/HER2−) on an ER+ breast cancer.

It may be appreciated that when an element is specified then all isotopes of that element are also covered. For instance, the term “H” or “hydrogen” may be understood to also cover deuterium and tritium. In particular, deuterated analogues of the compounds may be used in imaging and/or metabolism studies.

The term “alkyl”, as used herein, unless otherwise specified, refers to a saturated straight or branched hydrocarbon. In certain embodiments, the alkyl group is a primary, secondary, or tertiary hydrocarbon. In certain embodiments, the alkyl group includes one to twelve carbon atoms, i.e. C₁-C₁₂ alkyl, or one to six carbon atoms, i.e. C₁-C₆ alkyl. C₁-C₆ alkyl includes for example methyl, ethyl, n-propyl, isopropyl, butyl, pentyl, hexyl, isobutyl, sec-butyl, tert-butyl, isopentyl, neopentyl, and isohexyl. An alkyl group can be unsubstituted or substituted with one or more of halogen, CN, OR¹⁴, SR¹⁴, NR¹⁴R¹⁵ or SX³₅, wherein R¹⁴ and R¹⁵ are H, optionally fluorinated C₁-C₁₅ alkyl, optionally fluorinated C₂-C₁₅ alkenyl or optionally fluorinated C₂-C₁₅ alkynyl and X³ is a halogen. In some embodiments, an alkyl group is unsubstituted or substituted with fluorine, SF₅, L³CF₃, L³CHF₂ or L³CH₂F, wherein L³ is absent or is O or S.

“Alkenyl” refers to olefinically unsaturated hydrocarbon groups which can be unbranched or branched. In certain embodiments, the alkenyl group has 2 to 6 carbons, i.e. it is a C₂-C₆ alkenyl. C₂-C₆ alkenyl includes for example vinyl, allyl, propenyl, butenyl, pentenyl and hexenyl. An alkenyl group can be unsubstituted or substituted with one or more of halogen, CN, OR¹⁴, SR¹⁴, NR¹⁴R¹⁵ or SX³₅, wherein R¹⁴ and R¹⁵ are H, optionally fluorinated C₁-C₁₅ alkyl, optionally fluorinated C₂-C₁₅ alkenyl or optionally fluorinated C₂-C₁₅ alkynyl and X³ is a halogen. In some embodiments, an alkenyl group is unsubstituted or substituted with fluorine, SF₅, L³CF₃, L³CHF₂ or L³CH₂F, wherein L³ is absent or is O or S.

“Alkynyl” refers to acetylenically unsaturated hydrocarbon groups which can be unbranched or branched. In certain embodiments, the alkynyl group has 2 to 6 carbons, i.e. it is a C₂-C₆ alkynyl. C₂-C₆ alkynyl includes for example propargyl, propynyl, butynyl, pentynyl and hexynyl. An alkynyl group can be unsubstituted or substituted with one or more of halogen, CN, OR¹⁴, SR¹⁴, NR¹⁴R¹⁵ or SX³₅, wherein R¹⁴ and R¹⁵ are H, optionally fluorinated C₁-C₁₅ alkyl, optionally fluorinated C₂-C₁₅ alkenyl or optionally fluorinated C₂-C₁₅ alkynyl and X³ is a halogen. In some embodiments, an alkynyl group is unsubstituted or substituted with fluorine, SF₅, L³CF₃, L³CHF₂ or L³CH₂F, wherein L³ is absent or is O or S.

The term “alkylene”, as used herein, unless otherwise specified, refers to a bivalent saturated straight or branched hydrocarbon. In certain embodiments, the alkylene group is a primary, secondary, or tertiary hydrocarbon. In certain embodiments, the alkylene group includes one to twelve carbon atoms, i.e. C₁-C₁₂ alkylene, or one to six carbon atoms, i.e. C₁-C₆ alkylene. C₁-C₆ alkylene includes for example methylene, ethylene, n-propylene and isopropylene, butylene, pentylene, hexylene, isobutylene, sec-butylene, tert-butylene, isopentylene, neopentylene, and isohexylene. An alkylene group can be unsubstituted or substituted with one or more of halogen, CN, OR¹⁴, SR¹⁴, NR¹⁴R¹⁵ or SX³₅, wherein R¹⁴ and R¹⁵ are H, optionally fluorinated C₁-C₁₅ alkyl, optionally fluorinated C₂-C₁₅ alkenyl or optionally fluorinated C₂-C₁₅ alkynyl and X³ is a halogen. In some embodiments, an alkylene group is unsubstituted or substituted with fluorine, SF₅, L³CF₃, L³CHF₂ or L³CH₂F, wherein L³ is absent or is O or S.

The term “alkylyne”, as used herein, unless otherwise specified, refers to a bivalent unsaturated straight or branched hydrocarbon. In certain embodiments, the alkylyne group is a primary, secondary, or tertiary hydrocarbon. In certain embodiments, the alkylyne group includes two to twelve carbon atoms, i.e. C₂-C₁₂ alkylyne, two to six carbon atoms, i.e. C₂-C₆ alkylyne. C₂-C₆ alkylyne includes for example ethylyne, propylyne, butylyne, pentylyne or hexylyne. An alkylyne group can be unsubstituted or substituted with one or more of halogen, CN, OR¹⁴, SR¹⁴, NR¹⁴R¹⁵ or SX³₅, wherein R¹⁴ and R¹⁵ are H, optionally fluorinated C₁-C₁₅ alkyl, optionally fluorinated C₂-C₁₅ alkenyl or optionally fluorinated C₂-C₁₅ alkynyl and X³ is a halogen. In some embodiments, an alkylyne group is unsubstituted or substituted with fluorine, SF₅, L³CF₃, L³CHF₂ or L³CH₂F, wherein L³ is absent or is O or S.

“Aryl” refers to an aromatic 6 to 12 membered hydrocarbon group. Examples of a C₆-C₁₂ aryl group include, but are not limited to, phenyl, α-naphthyl, β-naphthyl, biphenyl, tetrahydronaphthyl and indanyl. An aryl group can be unsubstituted or substituted with one or more of optionally substituted C₁-C₁₅ alkyl, optionally substituted C₂-C₁₅ alkenyl, optionally substituted C₂-C₁₅ alkynyl, halogen, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶.

“Heteroaryl” refers to a monocyclic or bicyclic aromatic 5 to 10 membered ring system in which at least one ring atom is a heteroatom. The, or each, heteroatom may be independently selected from the group consisting of oxygen, sulfur and nitrogen. Examples of 5 to 10 membered heteroaryl groups include furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3-benzodioxole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. Bicyclic 5 to 10 membered heteroaryl groups include those where a phenyl, pyridine, pyrimidine, pyrazine or pyridazine ring is fused to a 5 or 6-membered monocyclic heteroaryl ring. A heteroaryl group can be unsubstituted or substituted with one or more of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶. In some embodiments, a heteroatom may be substituted with one of the substituents. For instance, the heteroaryl may be a pyridine and it may be substituted with an O to provide a pyridine-N-oxide.

“Heterocycle” or “heterocyclyl” refers to a 3 to 8 membered monocyclic, bicyclic or bridged molecules in which at least one ring atom is a heteroatom. The or each heteroatom may be independently selected from the group consisting of oxygen, sulfur and nitrogen. A heterocycle may be saturated or partially saturated. Exemplary 3 to 8 membered heterocyclyl groups include but are not limited to aziridine, oxirane, oxirene, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, morpholine, piperazine, thiane, thiine, azepane, diazepane, oxazine. A heterocyclyl group can be unsubstituted or substituted with one or more of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶.

Pharmaceutically acceptable salts include any salt of a compound of formula (I) provided herein which retains its biological properties and which is not toxic or otherwise undesirable for pharmaceutical use. The pharmaceutically acceptable salt may be derived from a variety of organic and inorganic counter-ions well known in the art.

The pharmaceutically acceptable salt may comprise an acid addition salt formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2,2,2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids. Alternatively, the pharmaceutically acceptable salt may comprise a base addition salt formed when an acidic proton present in the parent compound is either replaced by a metal ion, e.g., an alkali metal ion, an alkaline earth ion, an aluminium ion, alkali metal or alkaline earth metal hydroxides, such as sodium, potassium, calcium, magnesium, aluminium, lithium, zinc, and barium hydroxide, or coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

Accordingly, the salt may comprise a group I or a group II metal salt, i.e. an alkali metal salt or an alkaline earth metal salt. Accordingly, the salt may comprise a lithium salt, a sodium salt, a potassium salt, a beryllium salt, a magnesium salt or a calcium salt.

A pharmaceutically acceptable solvate refers to a compound of formula (I), or a salt thereof, that further includes a stoichiometric or non-stoichiometric amount of solvent bound by non-covalent intermolecular forces. Where the solvent is water, the solvate is a hydrate.

In one embodiment, n is 1. X¹ may be CR². Accordingly, the compound may be a compound of formula (II):

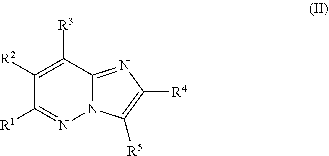

R² to R⁴ may each independently be hydrogen, a halogen, an optionally substituted C₁-C₆ alkyl, an optionally substituted C₂-C₆ alkenyl or an optionally substituted C₂-C₆ alkynyl. The halogen may be fluorine, chlorine, bromine or iodine, and is preferably fluorine. Accordingly, R² to R⁴ may each independently be hydrogen, fluorine or optionally substituted methyl.

In embodiments where at least one of R² to R⁴ are an optionally substituted alkyl, an optionally substituted alkenyl or an optionally substituted alkynyl, the or each alkyl, alkenyl or alkynyl is preferably independently unsubstituted or substituted with a halogen. The halogen may be fluorine, chlorine, bromine or iodine, and is preferably fluorine. Accordingly, one or more of R² to R⁴ may be a methyl or a halogenated methyl. In some embodiments, one or more of R² to R⁴ may be CH₃, CH₂F, CHF₂ or CF₃. Most preferably, the or each alkyl, alkenyl or alkynyl is unsubstituted.

In a preferred embodiment, R² to R⁴ are each H.

Accordingly, in a preferred embodiment, the compound is a compound of formula (II):

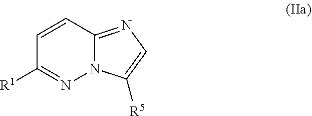

R¹ may be an optionally substituted C₆-C₁₂ aryl, an optionally substituted 5 to 10 membered heteroaryl, an optionally substituted 3 to 10 membered heterocycyl or a halogen. R¹ may be an optionally substituted phenyl, an optionally substituted thiophenyl, an optionally substituted thiazolyl, an optionally substituted tetrazolyl, an optionally substituted triazolyl, an optionally substituted pyridinyl, an optionally substituted pyridazinyl, an optionally substituted pyrimidinyl, an optionally substituted triazinyl, an optionally substituted 1,3-benzodioxolyl, an optionally substituted tetrahydropyranyl, an optionally substituted dihydropyranyl, an optionally substituted morpholinyl or chlorine.

R¹ is preferably an optionally substituted 5 or 6 membered heteroaryl. Preferably, R¹ is to an optionally substituted thiophenyl, an optionally substituted thiazolyl, an optionally substituted tetrazolyl, an optionally substituted triazolyl, an optionally substituted pyridinyl, an optionally substituted pyridazinyl, an optionally substituted pyrimidinyl or an optionally substituted triazinyl.

More preferably, R¹ is an optionally substituted 6 membered heteroaryl, and most preferably is an optionally substituted pyridinyl or an optionally substituted pyridazinyl.

R¹ may be:

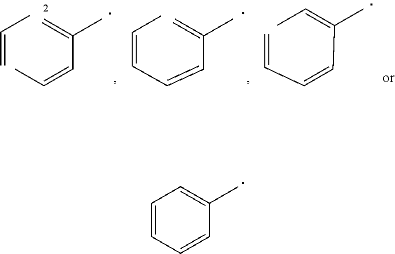

wherein X² is N or CR⁹; and

R⁹ to R¹³ are independently optionally substituted C₁-C₁₅ alkyl, optionally substituted C₂-C₁₅ alkenyl, optionally substituted C₂-C₁₅ alkynyl, halogen, OR⁶, NR⁶R⁷, CONR⁶R⁷, CN, COR⁶, COOR⁶, NO₂, NR⁶COR⁷, OC(O)OR⁶, OC(O)NR⁶R⁷ and OC(O)R⁶.

Preferably, R⁹ to R¹³ are independently optionally substituted C₁-C₆ alkyl, optionally substituted C₂-C₆ alkenyl, optionally substituted C₂-C₆ alkynyl, halogen, OR⁶, CONR⁶R⁷, NR⁶COR⁷, NR⁶SO₂R⁷ or CN, wherein R⁶ and R⁷ are H, optionally substituted C₁-C₆ alkyl, optionally substituted C₂-C₆ alkenyl or optionally substituted C₂-C₆ alkynyl. More preferably, R⁹ to R¹³ are independently optionally substituted C₁-C₃ alkyl, optionally substituted C₂-C₃ alkenyl, optionally substituted C₂-C₃ alkynyl, fluorine, chlorine, OR⁶, CONR⁶R⁷, NR⁶COR⁷, NR⁶SO₂R⁷ or CN, wherein R⁶ and R⁷ are H, optionally substituted C₁-C₃ alkyl, optionally substituted C₂-C₃ alkenyl or optionally substituted C₂-C₃ alkynyl. Even more preferably, R⁹ to R¹³ are independently optionally substituted C₁-C₃ alkyl, optionally substituted C₂-C₃ alkenyl, optionally substituted C₂-C₃ alkynyl, fluorine, chlorine, OR⁶, NHCOCH₃, NHSO₂CH₃ or CN, wherein R⁶ is H, optionally substituted C₁-C₃ alkyl, optionally substituted C₂-C₃ alkenyl or optionally substituted C₂-C₃ alkynyl. Most preferably, R⁹ to R¹³ are independently optionally substituted methyl, fluorine, OR⁶, NHCOCH₃, NHSO₂CH₃ or CN, wherein R⁶ is H or optionally substituted methyl. R⁹ to R¹³ may each independently be H or methyl. In some embodiments, R⁹ to R¹³ are each H.

If a preferred embodiment, R¹ is:

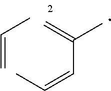

Preferably, X² is CR⁹.

R¹⁰ and R¹² may each independently be H or a methyl. In some embodiments, R¹⁰ and R¹² are each H.

In some embodiments, R⁹ and R¹³ are H.

Accordingly, R¹ may be Cl, phenyl,

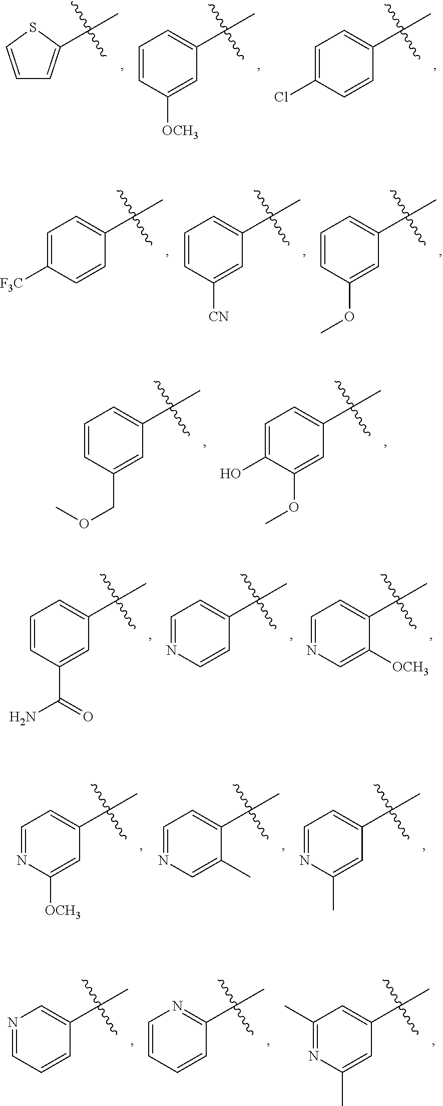

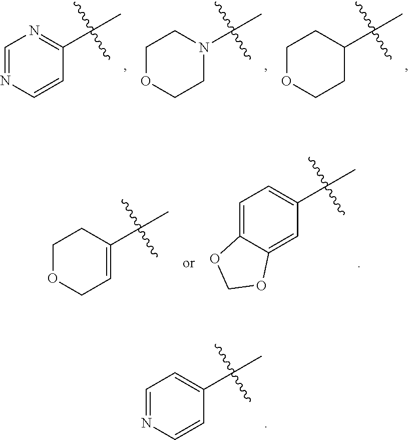 [SMILES: *c1ccncn1 ; *N1CCOCC1 ; *C1CCOCC1 ; *C1=CCOCC1 ; *c1ccc2c(c1)OCO2 ; *c1ccncc1]

Preferably, R¹ is

R⁵ may be an optionally substituted C₆-C₁₂ aryl, an optionally substituted 5 to 10 membered heteroaryl or an optionally substituted 3 to 10 membered heterocycyl. R⁵ may be an optionally substituted phenyl, an optionally substituted thiophenyl, an optionally substituted thiazolyl, an optionally substituted tetrazolyl, an optionally substituted triazolyl, an optionally substituted pyridinyl, an optionally substituted pyridazinyl, an optionally substituted pyrimidinyl, an optionally substituted triazinyl, an optionally substituted 1,3-benzodioxolyl, an optionally substituted tetrahydropyranyl, an optionally substituted dihydropyranyl, an optionally substituted morpholinyl or chlorine.

R⁵ is preferably an optionally substituted phenyl or an optionally substituted 5 or 6 membered heteroaryl. Preferably, R⁵ is and optionally substituted phenyl, an optionally substituted thiophenyl, an optionally substituted thiazolyl, an optionally substituted tetrazolyl, an optionally substituted triazolyl, an optionally substituted pyridinyl, an optionally substituted pyridazinyl, an optionally substituted pyrimidinyl or an optionally substituted triazinyl. More preferably, R⁵ is an optionally substituted phenyl.

R⁵ may be an unsubstituted phenyl.

In a preferred embodiment, R⁵ is a phenyl or a 5 or 6 membered heteroaryl substituted with one or more substituents selected from the group consisting of optionally substituted C₁-C₆ alkyl, optionally substituted C₂-C₆ alkenyl, optionally substituted C₂-C₆ alkynyl, halogen, OR⁶, SR⁶, COR⁶ and CONR⁶R⁷, wherein R⁶ and R⁷ are H, optionally substituted C₁-C₆ alkyl, optionally substituted C₂-C₆ alkenyl or optionally substituted C₂-C₆ alkynyl. More preferably, R⁵ is a phenyl or a 5 or 6 membered heteroaryl substituted with one or more substituents selected from the group consisting of optionally substituted C₁-C₃ alkyl, optionally substituted C₂-C₃ alkenyl, optionally substituted C₂-C₃ alkynyl, halogen, OR⁶, SR⁶, COR⁶ and CONR⁶R⁷, wherein R⁶ and R⁷ are H, optionally substituted C₁-C₃ alkyl, optionally substituted C₂-C₃ alkenyl or optionally substituted C₂-C₃ alkynyl. Even more preferably, R⁵ is a phenyl or a 5 or 6 membered heteroaryl substituted with one or more substituents selected from the group consisting of optionally substituted methyl, fluorine, chlorine, OR⁶, SR⁶, COR⁶ and CONR⁶R⁷, wherein R⁶ is H, an optionally substituted methyl or an optionally substituted ethyl. R⁵ may be a phenyl substituted with one or more substituents selected from the group consisting of CH₃, CH₂F, CHF₂, CF₃, CH₂OH, fluorine, chlorine, OR⁶, SR⁶, COR⁶ and CONR⁶R⁷, wherein R⁶ is H, CH₃, CH₂F, CHF₂, CF₃ or CH₂CH₂N(CH₃)₂. In a most preferred embodiment, R⁵ is a phenyl substituted with OCF₃.

R⁵ may be a phenyl substituted with one or two substituents. The substituent may be in the ortho, meta or para position. In some embodiments, the substituent is in the meta position.

R⁵ may be

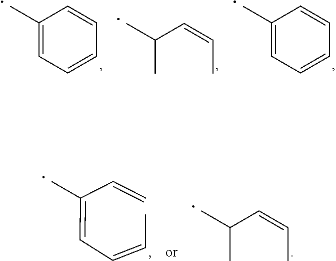

The compound of formula (I) may be a compound of formula (100) to (122):

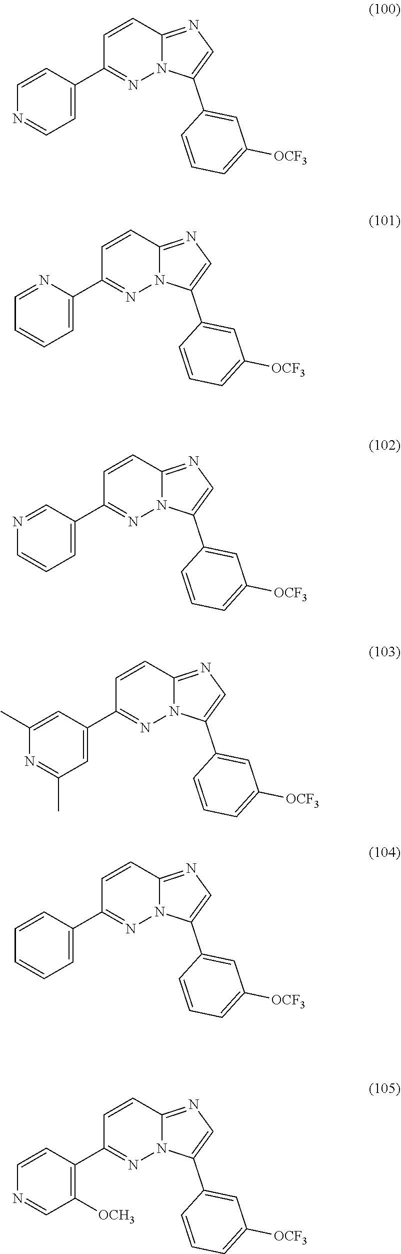 [SMILES: FC(F)(F)Oc1cccc(-c2cnc3ccc(-c4ccncc4)nn23)c1 ; FC(F)(F)Oc1cccc(-c2cnc3ccc(-c4ccccn4)nn23)c1 ; FC(F)(F)Oc1cccc(-c2cnc3ccc(-c4cccnc4)nn23)c1 ; Cc1cc(-c2ccc3ncc(-c4cccc(OC(F)(F)F)c4)n3n2)cc(C)n1 ; FC(F)(F)Oc1cccc(-c2cnc3ccc(-c4ccccc4)nn23)c1 ; COc1cnccc1-c1ccc2ncc(-c3cccc(OC(F)(F)F)c3)n2n1]

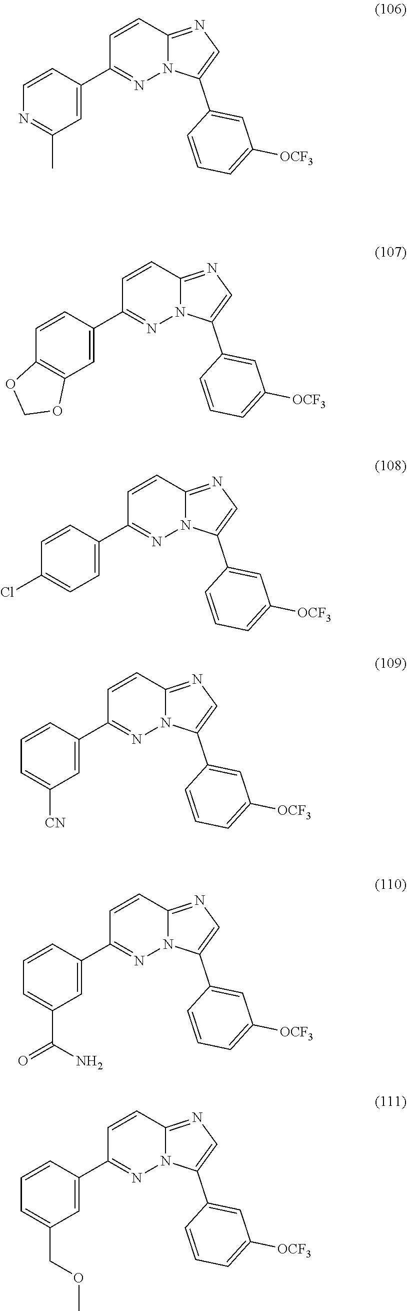 [SMILES: Cc1cc(-c2ccc3ncc(-c4cccc(OC(F)(F)F)c4)n3n2)ccn1 ; FC(F)(F)Oc1cccc(-c2cnc3ccc(-c4ccc5c(c4)OCO5)nn23)c1 ; FC(F)(F)Oc1cccc(-c2cnc3ccc(-c4ccc(Cl)cc4)nn23)c1 ; N#Cc1cccc(-c2ccc3ncc(-c4cccc(OC(F)(F)F)c4)n3n2)c1 ; NC(=O)c1cccc(-c2ccc3ncc(-c4cccc(OC(F)(F)F)c4)n3n2)c1 ; COCc1cccc(-c2ccc3ncc(-c4cccc(OC(F)(F)F)c4)n3n2)c1]

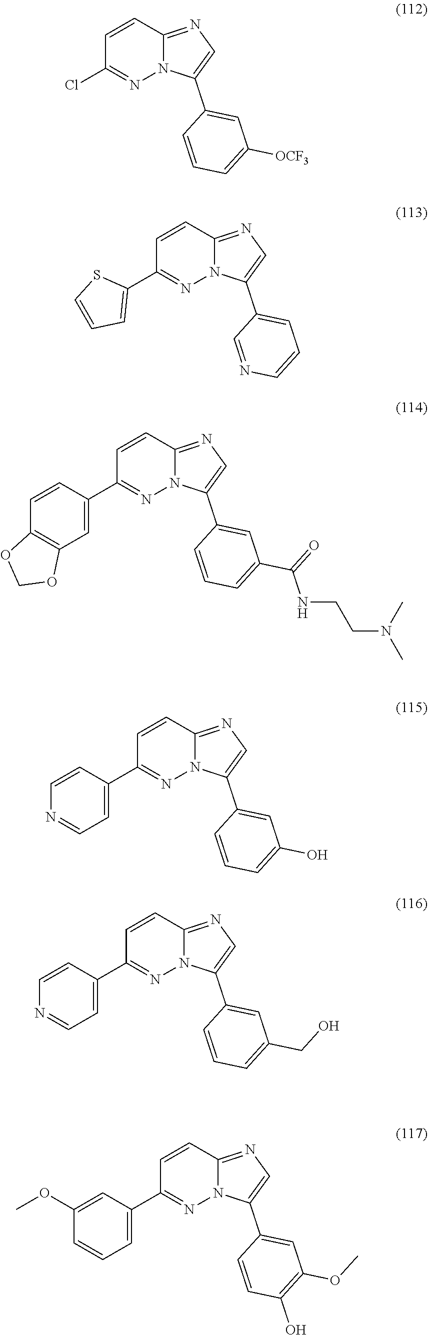

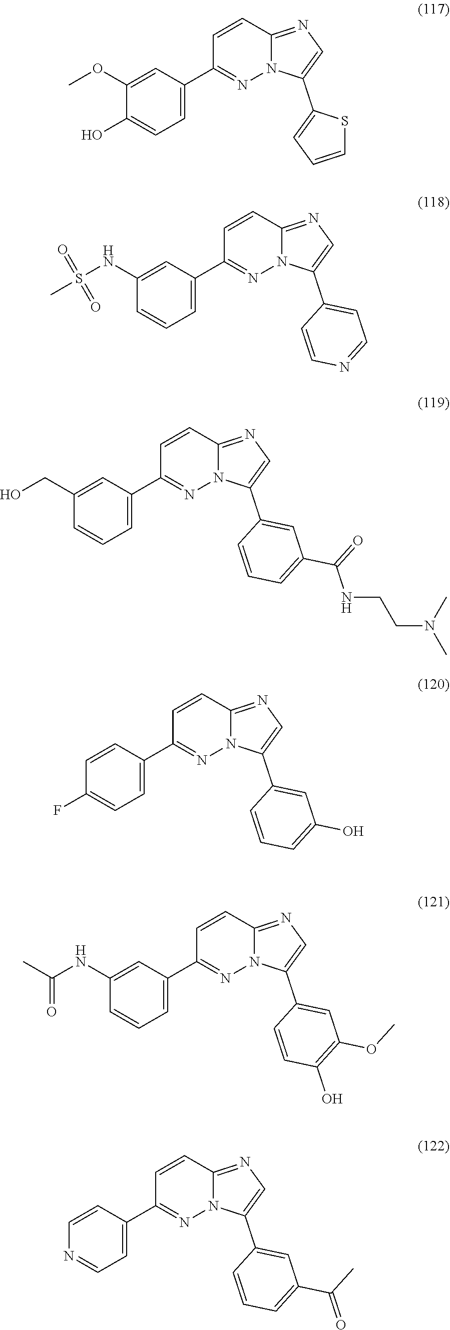

In some embodiments, the compound is a compound of formula (100).

In one embodiment, n is o. X¹ may be S. Accordingly, the compound may be a compound of formula (III):

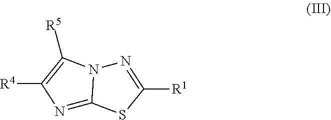

Preferably, R¹ is L¹L²R⁸. Accordingly, the compound may be a compound of formula (IIIa):

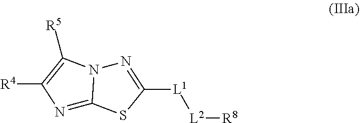

L¹ is preferably O, S or NR⁶, and more preferably is NR⁶. R⁶ is preferably H.

L² is preferably an optionally substituted C₁ to C₁₀ alkylene or an optionally substituted C₂ to C₁₀ alkylyne, more preferably is an optionally substituted C₁ to C₅ alkylene or an optionally substituted C₂ to C₅ alkylyne and most preferably is an optionally substituted C₁ to C₃ alkylene or an optionally substituted C₂ to C₃ alkylyne. In a preferred embodiment, L² is —CH₂—.

R⁸ is preferably an optionally substituted C₆-C₁₂ aryl, an optionally substituted 5 to 10 membered heteroaryl, an optionally substituted 3 to 10 membered heterocycyl. R⁸ is preferably an optionally substituted phenyl or an optionally substituted 5 or 6 membered heteroaryl.

R⁸ may be an optionally substituted phenyl, an optionally substituted thiophenyl, an optionally substituted thiazolyl, an optionally substituted tetrazolyl, an optionally substituted triazolyl, an optionally substituted pyridinyl, an optionally substituted pyridazinyl, an optionally substituted pyrimidinyl, an optionally substituted triazinyl, an optionally substituted 1,3-benzodioxolyl, an optionally substituted tetrahydropyranyl, an optionally substituted dihydropyranyl or an optionally substituted morpholinyl.

More preferably, R⁸ is an optionally substituted phenyl, an optionally substituted pyridinyl or an optionally substituted pyridazinyl.

R⁸ may be phenyl or

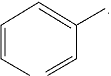

R⁴ may be hydrogen, a halogen, an optionally substituted C₁-C₆ alkyl, an optionally substituted C₂-C₆ alkenyl or an optionally substituted C₂-C₆ alkynyl. The halogen may be fluorine, chlorine, bromine or iodine, and is preferably fluorine. Accordingly, R⁴ be hydrogen, fluorine or optionally substituted methyl. Preferably, R⁴ is hydrogen.

R⁵ is preferably an optionally substituted C₆-C₁₂ aryl, an optionally substituted 5 to 10 membered heteroaryl, an optionally substituted 3 to 10 membered heterocycyl. R⁵ is preferably an optionally substituted phenyl or an optionally substituted 5 or 6 membered heteroaryl.

R⁵ may be an optionally substituted phenyl, an optionally substituted thiophenyl, an optionally substituted thiazolyl, an optionally substituted tetrazolyl, an optionally substituted triazolyl, an optionally substituted pyridinyl, an optionally substituted pyridazinyl, an optionally substituted pyrimidinyl, an optionally substituted triazinyl, an optionally substituted 1,3-benzodioxolyl, an optionally substituted tetrahydropyranyl, an optionally substituted dihydropyranyl or an optionally substituted morpholinyl.

R⁵ may be an unsubstituted phenyl or an unsubstituted 5 or 6 membered heteroaryl. Alternatively, R⁵ may be a phenyl or a 5 or 6 membered heteroaryl substituted with one or more substituents selected from the group consisting of optionally substituted C₁-C₆ alkyl, optionally substituted C₂-C₆ alkenyl, optionally substituted C₂-C₆ alkynyl, halogen, OR⁶ and SR⁶, wherein R⁶ is H, optionally substituted C₁-C₆ alkyl, optionally substituted C₂-C₆ alkenyl or optionally substituted C₂-C₆ alkynyl. More preferably, R⁵ is a phenyl or a 5 or 6 membered heteroaryl substituted with one or more substituents selected from the group consisting of optionally substituted C₁-C₃ alkyl, optionally substituted C₂-C₃ alkenyl, optionally substituted C₂-C₃ alkynyl, halogen, OR⁶ and SR⁶, wherein R⁶ is H, optionally substituted C₁-C₃ alkyl, optionally substituted C₂-C₃ alkenyl or optionally substituted C₂-C₃ alkynyl. Even more preferably, R⁵ is a phenyl or a 5 or 6 membered heteroaryl substituted with one or more substituents selected from the group consisting of optionally substituted methyl, fluorine, chlorine, OR⁶ and SR⁶, wherein R⁶ is H or an optionally substituted methyl. R⁵ may be a phenyl or a 5 or 6 membered heteroaryl substituted with one or more substituents selected from the group consisting of CH₃, CH₂F, CHF₂, CF₃, chlorine, fluorine, OR⁶ and SR⁶, wherein R⁶ is H, CH₃, CH₂F, CHF₂ or CF₃.

R⁵ may be a phenyl or a 6 membered heteroaryl substituted with one substituent. The substituent may be in the ortho, meta or para position. In some embodiments, the substituent is in the meta or para position.

Accordingly, R⁵ may be phenyl,

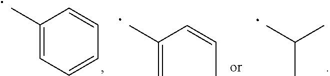

The compound of formula (I) may be a compound of formula (200) to (204):

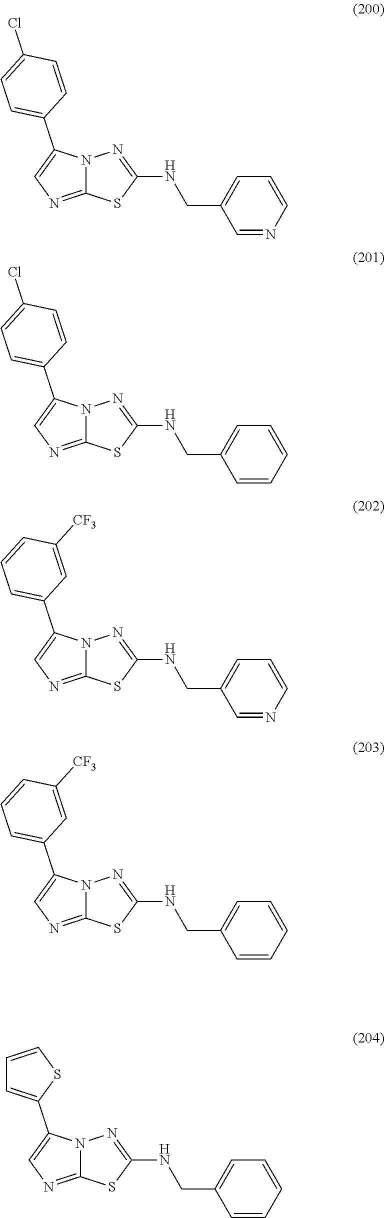 [SMILES: Clc1ccc(-c2cnc3sc(NCc4cccnc4)nn23)cc1 ; Clc1ccc(-c2cnc3sc(NCc4ccccc4)nn23)cc1 ; FC(F)(F)c1cccc(-c2cnc3sc(NCc4cccnc4)nn23)c1 ; FC(F)(F)c1cccc(-c2cnc3sc(NCc4ccccc4)nn23)c1 ; c1ccc(CNc2nn3c(-c4cccs4)cnc3s2)cc1]

It will be appreciated that the compound of formula (I) described herein, or a pharmaceutically acceptable salt or solvate thereof, may be used in a medicament which may be used in a monotherapy (i.e. use of the inhibitor alone), for treating, ameliorating, or preventing cancer. Alternatively, the inhibitor or a pharmaceutically acceptable salt or solvate thereof may be used as an adjunct to, or in combination with, known therapies for treating, ameliorating, or preventing cancer. The known therapy may be a known endocrine and/or chemo-therapy. For example, the compound of formula (I) may be used in combination with a tamoxifen or doxorubicin.

The compound of formula (I) may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising the compound of formula (I) may be used in a number of ways. Compositions comprising the compound of formula (I) may be administered by inhalation (e.g. intranasally). Compositions may also be formulated for topical use. For instance, creams or ointments may be applied to the skin.

The compound of formula (I) may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with the inhibitor used according to the invention is required and which would normally require frequent administration (e.g. at least daily injection).

The compound of formula (I) and compositions comprising the compound may be administered to a subject by injection into the blood stream or directly into a site requiring treatment, for example into a cancerous tumour or into the blood stream adjacent thereto. Injections may be intravenous (bolus or infusion) or subcutaneous (bolus or infusion), intradermal (bolus or infusion) or intramuscular (bolus or infusion).

In a preferred embodiment, the compound of formula (I) is administered orally. Accordingly, the compound of formula (I) may be contained within a composition that may, for example, be ingested orally in the form of a tablet, capsule or liquid.

It will be appreciated that the amount of the compound of formula (I) that is required is determined by its biological activity and bioavailability, which in turn depends on the mode of administration, the physiochemical properties of the compound of formula (I), and whether it is being used as a monotherapy, or in a combined therapy. The frequency of administration will also be influenced by the half-life of the compound of formula (I) within the subject being treated. Optimal dosages to be administered may be determined by those skilled in the art, and will vary with the particular inhibitor in use, the strength of the pharmaceutical composition, the mode of administration, and the advancement of the cancer. Additional factors depending on the particular subject being treated will result in a need to adjust dosages, including subject age, weight, sex, diet, and time of administration.

The compound of formula (I) may be administered before, during or after onset of the cancer to be treated. Daily doses may be given as a single administration. Alternatively, the compound of formula (I) is given two or more times during a day, and may be given twice a day.

Generally, a daily dose of between 0.01 μg/kg of body weight and 500 mg/kg of body weight of the compound of formula (I) may be used for treating, ameliorating, or preventing cancer. More preferably, the daily dose is between 0.01 mg/kg of body weight and 400 mg/kg of body weight, more preferably between 0.1 mg/kg and 200 mg/kg body weight, and most preferably between approximately 1 mg/kg and 100 mg/kg body weight.

A patient receiving treatment may take a first dose upon waking and then a second dose in the evening (if on a two dose regime) or at 3- or 4-hourly intervals thereafter. Alternatively, a slow release device may be used to provide optimal doses of the inhibitor according to the invention to a patient without the need to administer repeated doses.

Known procedures, such as those conventionally employed by the pharmaceutical industry (e.g. in vivo experimentation, clinical trials, etc.), may be used to form specific formulations comprising the inhibitor according to the invention and precise therapeutic regimes (such as daily doses of the inhibitor and the frequency of administration). The inventors believe that they are the first to describe a pharmaceutical composition for treating cancer, based on the use of the compound of formula (I).

Hence, in a fourth aspect, there is provided a pharmaceutical composition for treating cancer in a subject, the composition comprising a compound of formula (I) and a pharmaceutically acceptable vehicle, wherein the compound of formula (I) is:

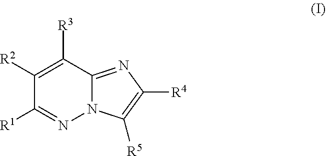

wherein R¹ is an optionally substituted C₆-C₁₂ aryl, an optionally substituted 5 to 10 membered heteroaryl, an optionally substituted 3 to 10 membered heterocycyl, L¹L²R⁸ or a halogen, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;

n is o and X¹ is S, O or NR²; or n is 1 and X¹ is CR² or N;

R² to R⁴ are independently hydrogen, a halogen, an optionally substituted C₁-C₁₅ alkyl, an optionally substituted C₂-C₁₅ alkenyl or an optionally substituted C₂-C₁₅ alkynyl;

R⁵ is an optionally substituted C₆-C₁₂ aryl, an optionally substituted 5 to 10 membered heteroaryl, an optionally substituted 3 to 10 membered heterocycyl, or L¹L²R⁸, wherein the aryl, heteroaryl or heterocycyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, OR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶; and

R⁶ and R⁷ are independently H, optionally substituted C₁-C₁₅ alkyl, optionally substituted C₂-C₁₅ alkenyl or optionally substituted C₂-C₁₅ alkynyl;

R⁸ is OR⁶, SR⁶, NR⁶R⁷, CONR⁶R⁷, CN, COOR⁶, NO₂, NR⁶COR⁷, NR⁶SO₂R⁷, OC(O)OR⁶, OC(O)NR⁶R⁷, OC(O)R⁶, an optionally substituted C₆-C₁₂ aryl, an optionally substituted 5 to 10 membered heteroaryl, an optionally substituted 3 to 10 membered heterocycyl, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;

L¹ is absent or is O, S or NR⁶; and

L² is absent or is an optionally substituted C₁ to C₁₅ alkylene or an optionally substituted C₂ to C₁₅ alkylyne;

or a pharmaceutically acceptable complex, salt, solvate, tautomeric form or polymorphic form thereof.

The pharmaceutical composition can be used in the therapeutic amelioration, prevention or treatment in a subject of cancer.

The pharmaceutical composition may further comprise a known therapy for treating, ameliorating, or preventing cancer.

The invention also provides, in a fifth aspect, a process for making the composition according to the fourth aspect, the process comprising contacting a therapeutically effective amount of a compound of formula (I), or a pharmaceutically acceptable complex, salt, solvate, tautomeric form or polymorphic form thereof, and a pharmaceutically acceptable vehicle.

A “subject” may be a vertebrate, mammal, or domestic animal. Hence, the compound of formula (I), compositions and medicaments according to the invention may be used to treat any mammal, for example livestock (e.g. a horse), pets, or may be used in other veterinary applications. Most preferably, however, the subject is a human being.

A “therapeutically effective amount” of the compound of formula (I) is any amount which, when administered to a subject, is the amount of drug that is needed to treat the cancer.

For example, the therapeutically effective amount of the compound of formula (I) used may be from about 0.01 mg to about 800 mg, and preferably from about 0.01 mg to about 500 mg. It is preferred that the amount of the compound of formula (I) is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents (i.e. the inhibitor) according to the invention. In tablets, the inhibitor may be mixed with a vehicle having the necessary compression properties in suitable proportions and compacted in the shape and size desired. The powders and tablets preferably contain up to 99% of the inhibitor. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The compound of formula (I) may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The compound of formula (I) may be prepared as a sterile solid composition that may be dissolved or suspended at the time of administration using sterile water, saline, or other appropriate sterile injectable medium.

The compound of formula (I) and compositions of the invention may be administered in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The compound of formula (I) used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

The inventors believe that they have identified novel compounds per se.

Accordingly, in accordance with a sixth aspect, there is provided a compound of formula (I):

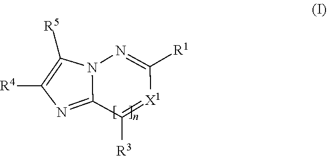

wherein R¹ is an optionally substituted C₆-C₁₂ aryl, an optionally substituted 5 to 10 membered heteroaryl, an optionally substituted 3 to 10 membered heterocycyl, L¹L²R⁸ or a halogen, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;

n is o and X¹ is S, O or NR²; or n is 1 and X¹ is CR² or N;

R² to R⁴ are independently hydrogen, a halogen, an optionally substituted C₁-C₁₅ alkyl, an optionally substituted C₂-C₁₅ alkenyl or an optionally substituted C₂-C₁₅ alkynyl; R⁵ is an optionally substituted C₆-C₁₂ aryl, an optionally substituted 5 to 10 membered heteroaryl, an optionally substituted 3 to 10 membered heterocycyl, or L¹L²R⁸, wherein the aryl, heteroaryl or heterocycyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, OR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶; and R⁶ and R⁷ are independently H, optionally substituted C₁-C₁₅ alkyl, optionally substituted C₂-C₁₅ alkenyl or optionally substituted C₂-C₁₅ alkynyl; R⁸ is OR⁶, SR⁶, NR⁶R⁷, CONR⁶R⁷, CN, COOR⁶, NO₂, NR⁶COR⁷, NR⁶SO₂R⁷, OC(O)OR⁶, OC(O)NR⁶R⁷, OC(O)R⁶, an optionally substituted C₆-C₁₂ aryl, an optionally substituted 5 to 10 membered heteroaryl, an optionally substituted 3 to 10 membered heterocycyl, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;

L¹ is absent or is O, S or NR⁶; and

L² is absent or is an optionally substituted C₁ to C₁₅ alkylene or an optionally substituted C₂ to C₁₅ alkylyne;

or a pharmaceutically acceptable complex, salt, solvate, tautomeric form or polymorphic form thereof,

wherein compounds of formula (100), (113) to (122) and (200) are excluded:

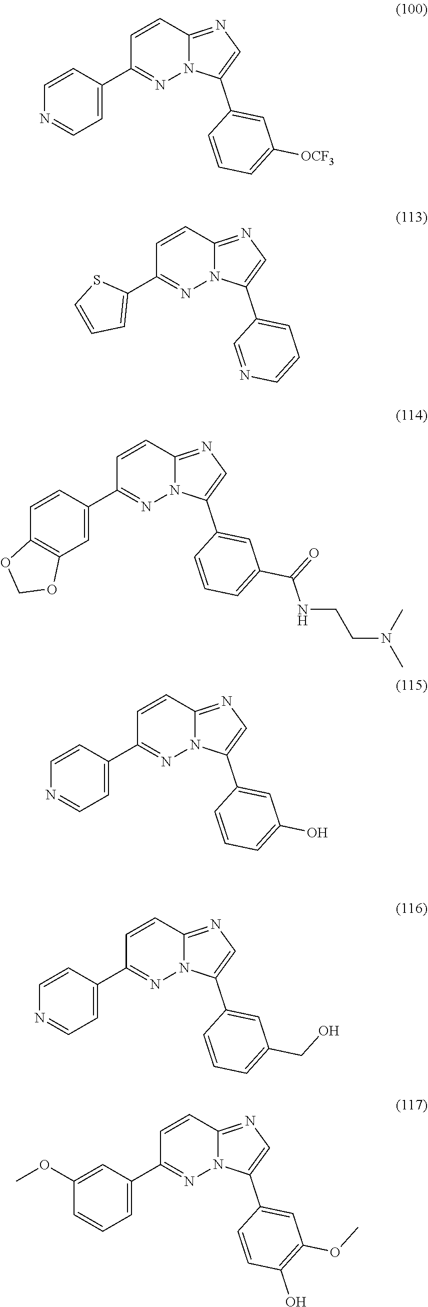

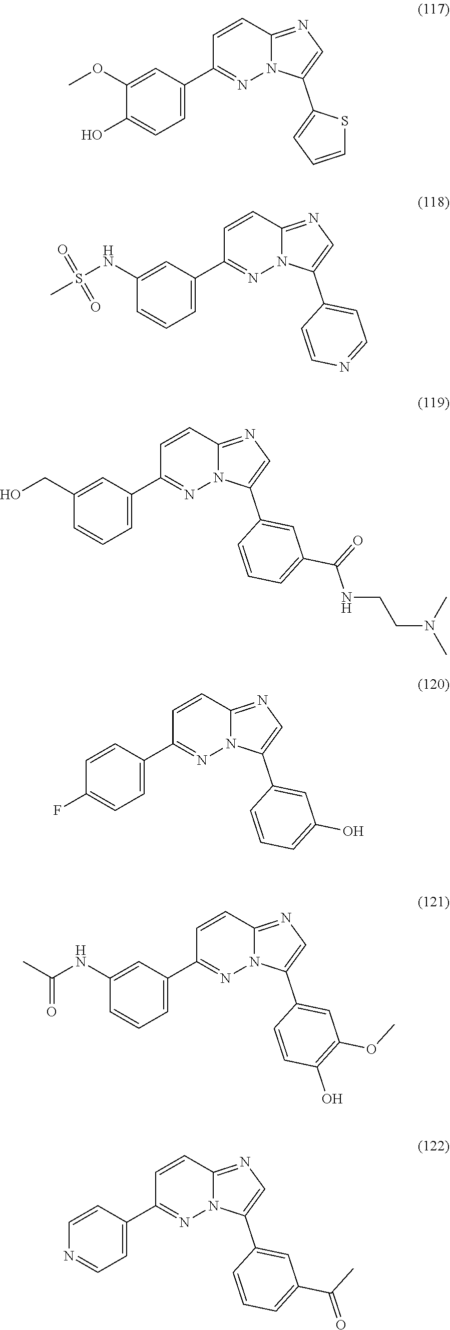

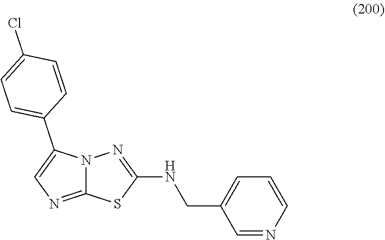 [SMILES: Clc1ccc(-c2cnc3sc(NCc4cccnc4)nn23)cc1]

The compound of the sixth aspect may be as defined in relation to the first and second aspects.

All features described herein (including any accompanying claims, abstract and drawings), and/or all of the steps of any method or process so disclosed, may be combined with any of the above aspects in any combination, except combinations where at least some of such features and/or steps are mutually exclusive.

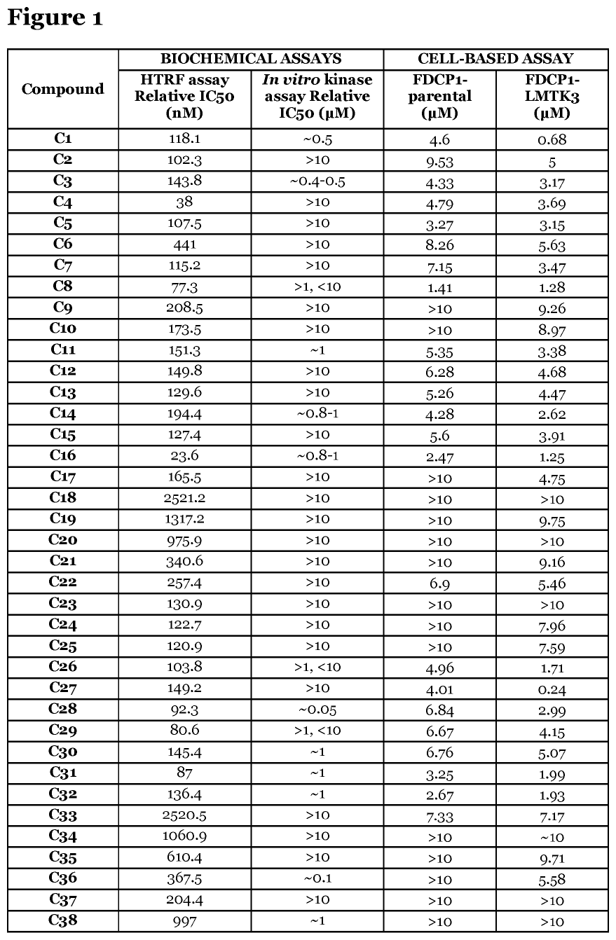

For a better understanding of the invention, and to show how embodiments of the same may be carried into effect, reference will now be made, by way of example, to the accompanying drawings, in which: FIG. 1 is a table summarizing the IC₅₀ values of the top 38 compounds assessed via the HTRF screening and by in vitro kinase assays as well as their EC₅₀ values in the FDCP1/FDCP1-LMTK3 cell-based model;

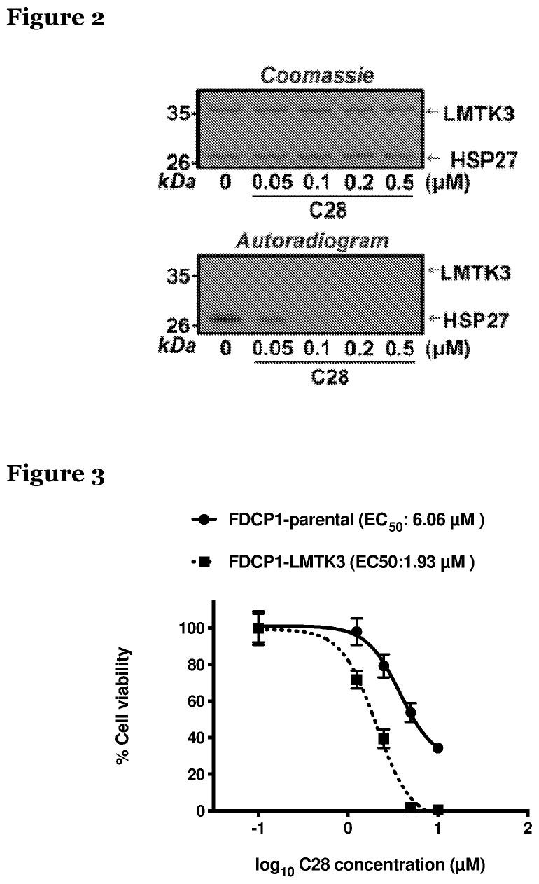

FIG. 2 shows how the IC₅₀ value for C28 against wt LMTK3-KD was determined by an in vitro kinase assay (performed in duplicate), with a starting dose of 0.5 μM down to 50 nM;

FIG. 3 is a graph showing growth inhibition for parental FDCP1 and transformed FDCP1-LMTK3 cells treated with increasing concentrations (1.25, 2.5, 5 and 10 μM) of C28. All error bars represent the mean±the standard deviation (SD) from 3 independent experiments;

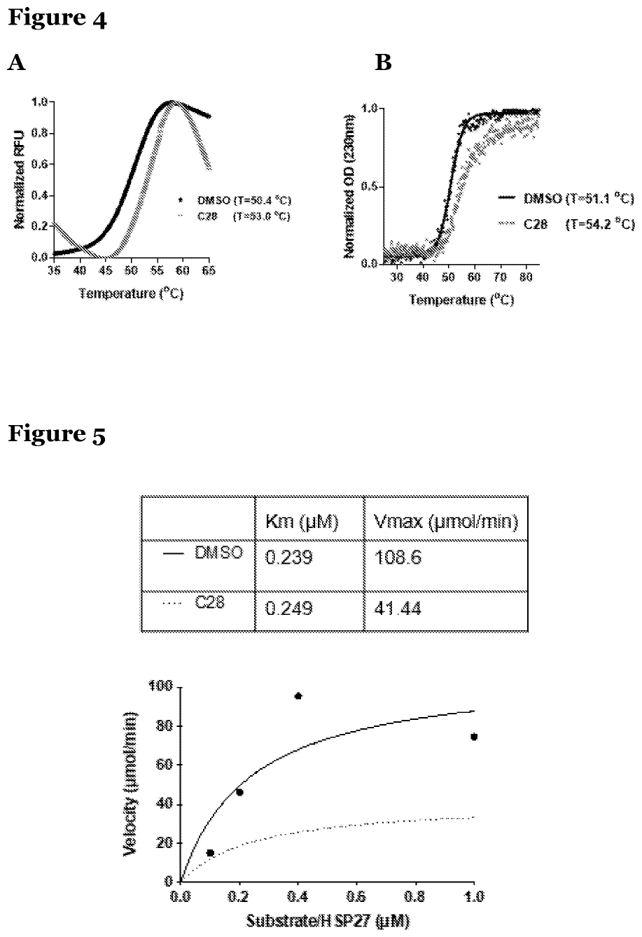

FIG. 4 shows graphs showing the characteristic thermal denaturation curves of LMTK3 (black) and LMTK3/C28 complex (red) as monitored by (A) differential scanning fluorimetry (DSF); and (B) circular dichroism (CD) spectroscopy, indicating the increased protein thermodynamic stability upon ligand binding. Apparent Tₘ values from DSF were determined from the maximum in the first derivative of the fluorescence with respect to the temperature, or the midpoint in the transition region by fitting a Boltzmann sigmoidal to the CD data. Experiments have been performed in triplicate;

FIG. 5 is a graph showing kinetic analysis of HSP27 phosphorylation by LMTK3 using a concentration of 2 μCi [γ-32] ATP in the absence or presence of 10 μM C28 inhibitor. Kinetic parameters were determined from non-linear regression fit of the initial reaction rates as a function of HSP27 concentration to the Michaelis-Menten equation using Prism 8;

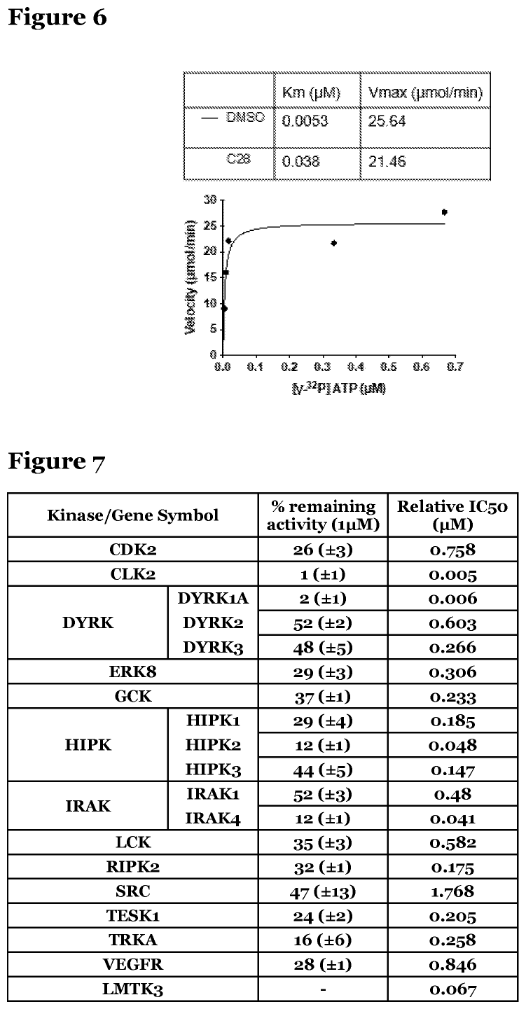

FIG. 6 is a graph showing kinetic analysis as a function of ATP concentration for 0.6 μM of HSP27 substrate, in the absence or presence of 10 μM C28 inhibitor. Kinetic parameters were determined from non-linear regression fit of the initial reaction rates as a function of ATP concentration to the Michaelis-Menten equation using Prism 8;

FIG. 7 shows results from a radioactive filter binding assay to examine the selectivity profile of C28 against 140 kinases at 1 μM concentration. The data is displayed as % activity remaining of assay duplicates with a standard deviation. Only the kinases showing a >50% decrease in their activity are shown. The relative IC₅₀ values of the top hits are also displayed. The screening was performed by MRC International Centre for Kinase Profiling unit (http://www.kinase-screen.mrc.ac.uk/services/premier-screen);

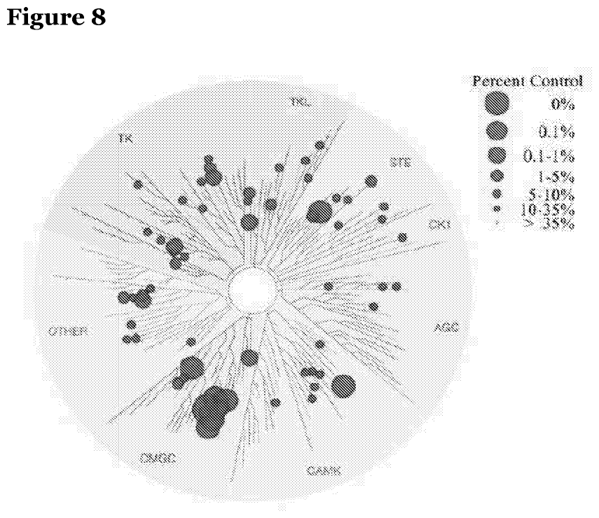

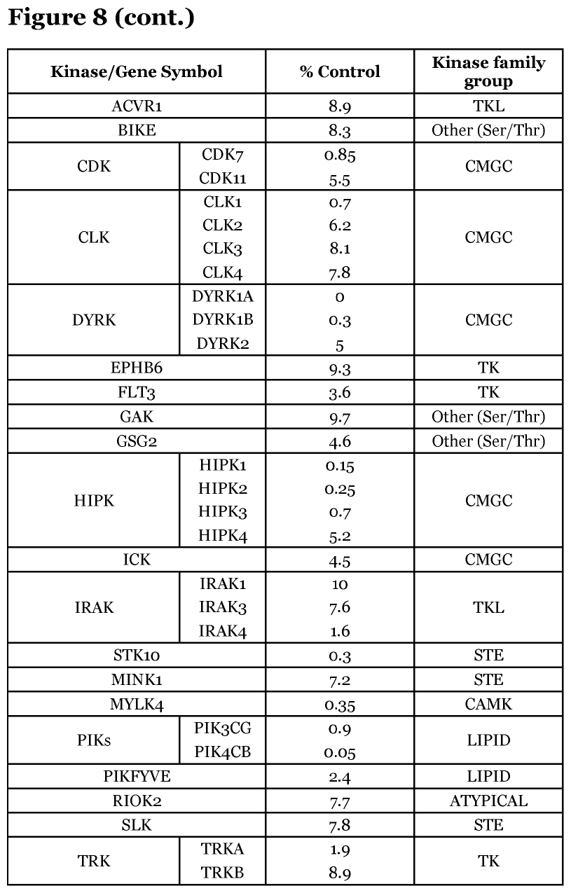

FIG. 8 provides a treespot interaction map (http://treespot.discoverx.com/) depicting the kinome phylogenetic grouping, with kinases interacting with C28 (5 μM) represented as red circles. The larger the diameter of the red circle, the higher the C28 binding affinity to the respective kinase. The list of kinases whose binding was inhibited by C28 to less than 10% of the negative control (DMSO) is shown. Lower numbers indicate stronger hits suggesting that these kinases represent the most probable hits to bind to C28. (Highlighted are the overlapping kinases that were identified in both the radioactive filter binding assay and the site-directed competition binding assay);

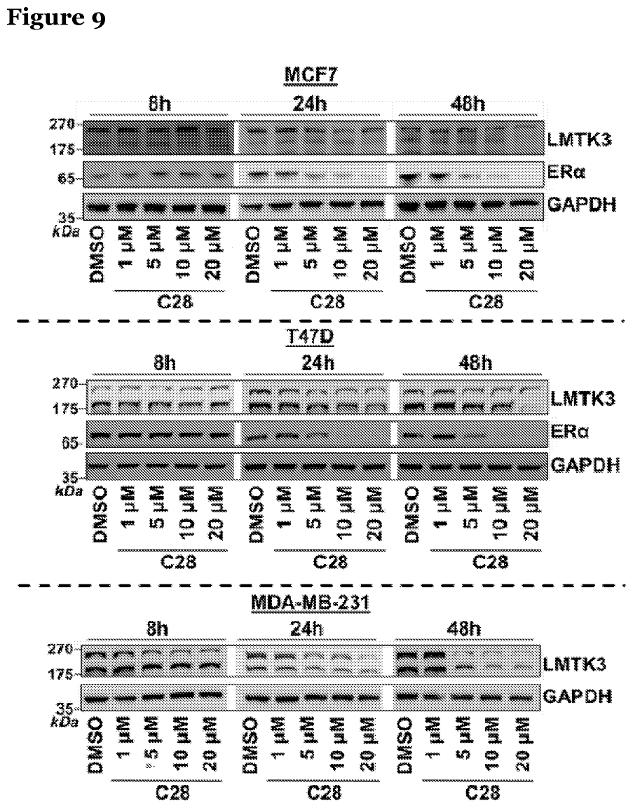

FIG. 9 shows Western blotting analysis of LMTK3 and ERα protein levels in MCF7, T47D and MDA-MB-231 cell lines following treatment with increasing concentrations of C28 at different time points;

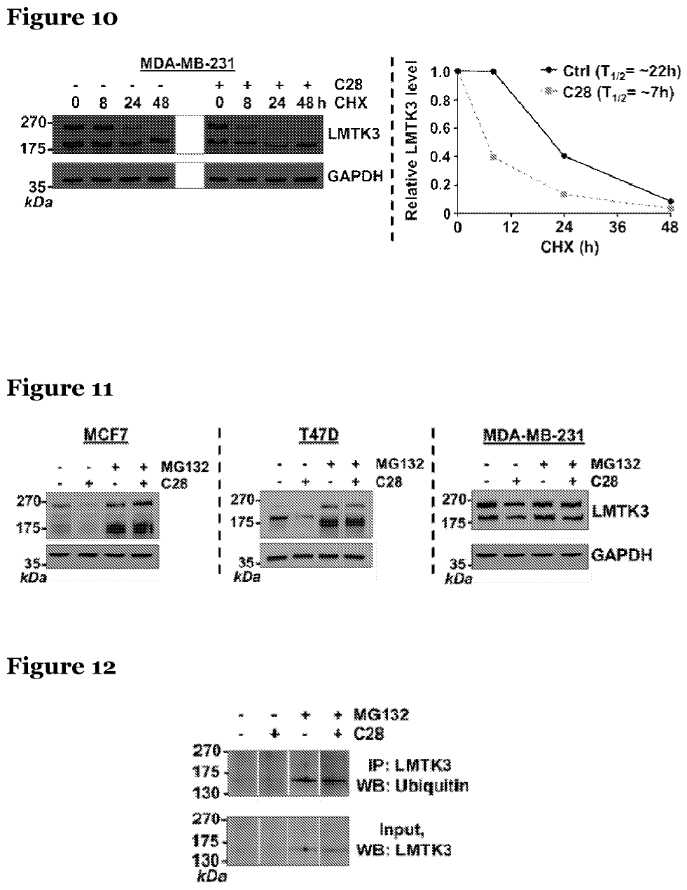

FIG. 10 shows the effects of C28 on LMTK3 protein half-life in MDA-MB-231 cells. Cells were treated with 100 μg/ml of Cyclohexamide (CHX) and 10 μM of C28 (or DMSO) for different time points. The relative LMTK3 protein levels (−/+C28) were calculated and plotted against the time of treatment with CHX;

FIG. 11 shows the effects of C28 on LMTK3 degradation in MCF7, T47D and MDA-MB-231 cells. Cells were treated with 10 μM of MG132 proteasome inhibitor (or DMSO) for 4 h and then with 10 μM of C28 (or DMSO) for 24 h, followed by Western blotting analysis of LMTK3 protein levels;

FIG. 12 shows the effects of C28 (10 μM; 24 h) on the polyubiquitination of LMTK3 in MCF7 cells. The respective input is shown in FIG. 11;

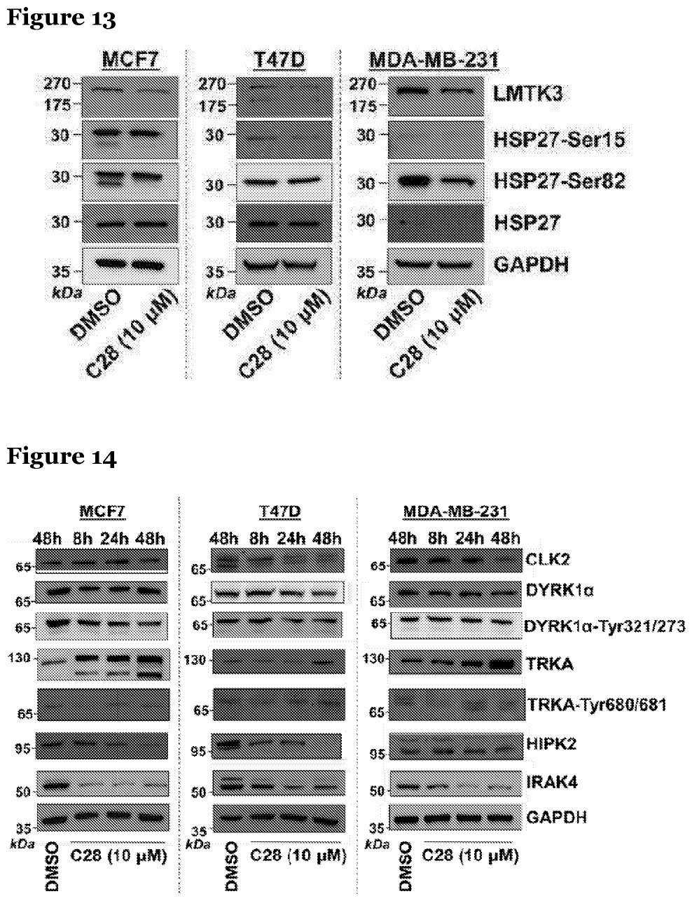

FIG. 13 shows Western blots of total and phospho-protein levels of HSP27 in MCF7, T47D and MDA-MB-231 cell lines following treatment with 10 μM of C28 for 24 h. Cells were treated with 25 μg/ml of anisomycin for 1 h to induce phosphorylation of HSP27;

FIG. 14 shows Western blots of different kinases' protein levels in MCF7, T47D and MDA-MB-231 cell lines following treatment with 10 μM of C28 at different time points;

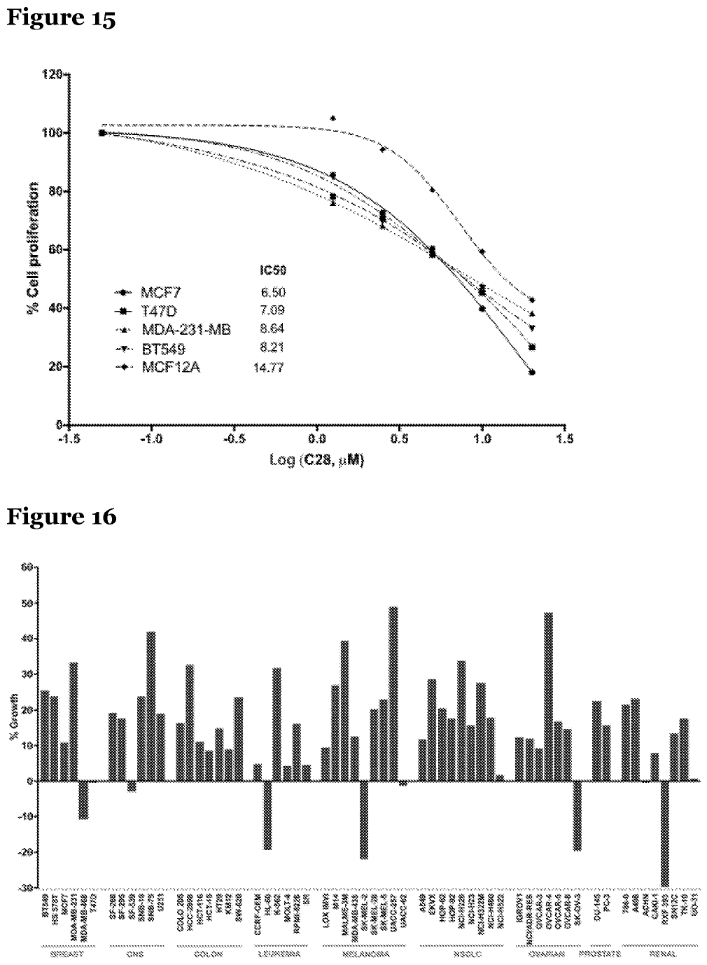

FIG. 15 is a graph showing the proliferation of normal and breast cancer cell lines following treatment with increasing concentrations (0, 1.25, 2.5, 5 and 10 μM) of C28 for 72 h. The IC₅₀ values are means from three independent experiments;

FIG. 16 shows the results of one-dose screening of C28 (10 μM; 24 h) on the NCI-60 panel of tumor cell lines. The % growth of C28-treated cells is shown. Negative values (<0%) represent lethality;

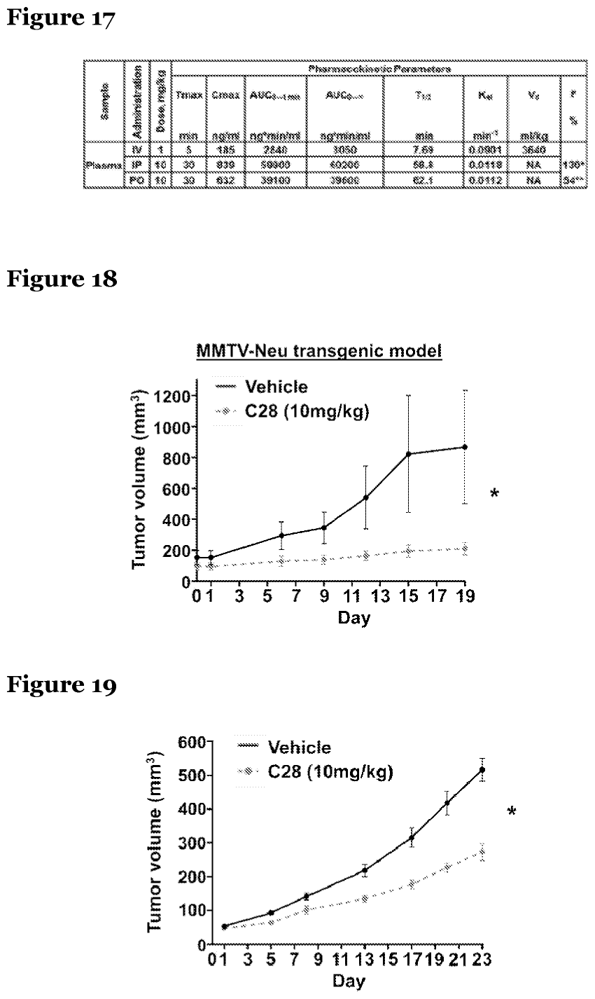

FIG. 17 is a table showing the pharmacokinetic parameters of C28 following a single IV, IP or PO dose administration in male BALB/c mice (n=4 per time point). Tₘₐₓ: time peak plasma concentration; Cₘₐₓ: maximum plasma concentration; AUC_{o→t min}: area under the plasma concentration-time curve to time of last measured concentration; AUC_{o→∞}: area under the plasma concentration-time curve extrapolated to infinity; T_{1/2}: plasma half-life; Kₑₗ: elimination rate constant; V_{d}: volume of distribution; F: peroral bioavailability. *: based on 1 mg/kg IV group. **: based on 5 mg/kg IV group. IV dose of 5 mg/kg was lethal for some mice and therefore the dose of the compound was decreased to 1 mg/kg. Calculated per oral bioavailability for C28 was 130% (based on the complete set of data from 1 mg/kg IV group). Possible reasons for observing F% higher than 100% with certain compounds and PK conditions, include ‘non-linear PK’ (metabolic saturation) at non-equal IV and PO doses and enterohepatic recirculation. The first possibility appears to be the case since the calculation based on the incomplete set of data from 5 mg/kg group gives a more realistic 54% number for peroral bioavailability;

FIG. 18 is a tumor growth chart showing results for in vehicle- and C28-treated groups (n=6 each) of MMTV-Neu transgenic mice. Unpaired t-test was performed using Prism 8 software. Results are expressed as mean±SEM; * P<0.05;

FIG. 19 is a tumor growth chart showing results for in vehicle- and C28-treated groups (n=14 each) of MDA-MB-231 mice xenografts;

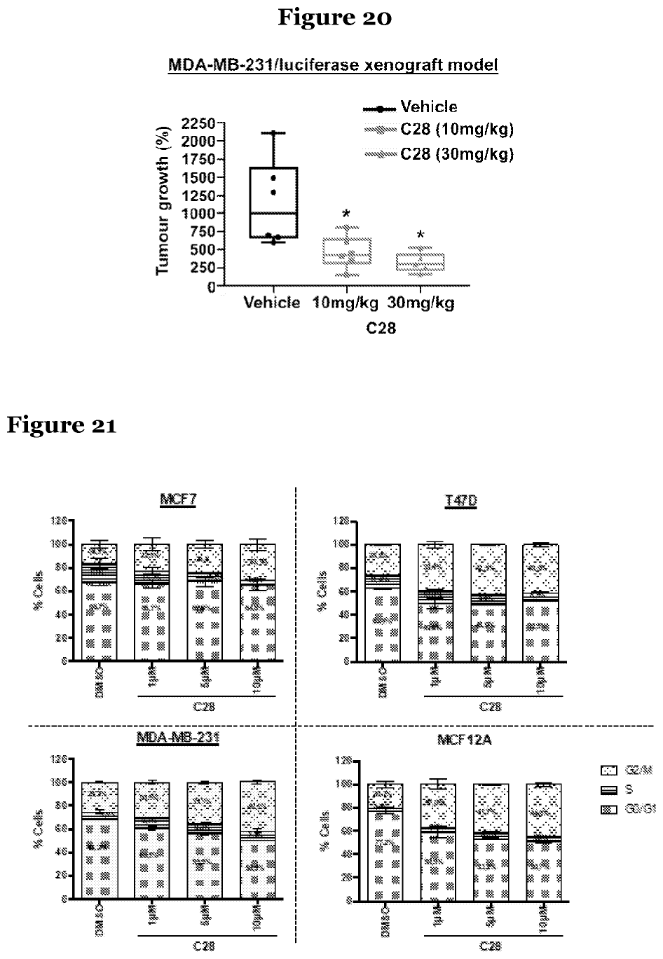

FIG. 20 is a box and whisker plots comparing vehicle (n=6)-treated and C28-treated (10 mg/kg/n=6 and 30 mg/kg/n=5) groups of MDA-MB-231-luciferase mice xenografts groups at Day 21. Unpaired t-test was performed using Prism 8 software. Results are expressed as mean±SEM; * P<0.05;

FIG. 21 shows the percentages of cells in G0/G1, S and G2/M phase are indicated. Results are expressed as mean±SEM. The experiment was performed 2 times;

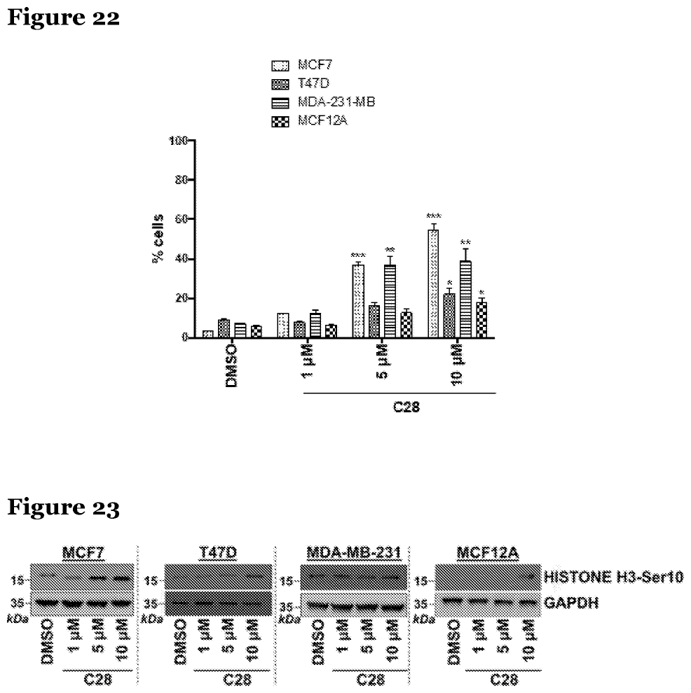

FIG. 22 shows the mitotic index as the percentage of mitotic cells over the total number of cells counted. The experiment was performed 2 times. ANOVA statistic test was performed using Prism 8 software. Results are expressed as mean±SEM; * P<0.05, ** P<0.01;

FIG. 23 shows Western blotting analysis of the phospho-histone H3 (Ser10) mitotic marker in MCF7, T47D, MDA-MB-231, MCF12A cell lines following treatment with increasing concentrations of C28 for 48 h. GADPH was used as loading control;

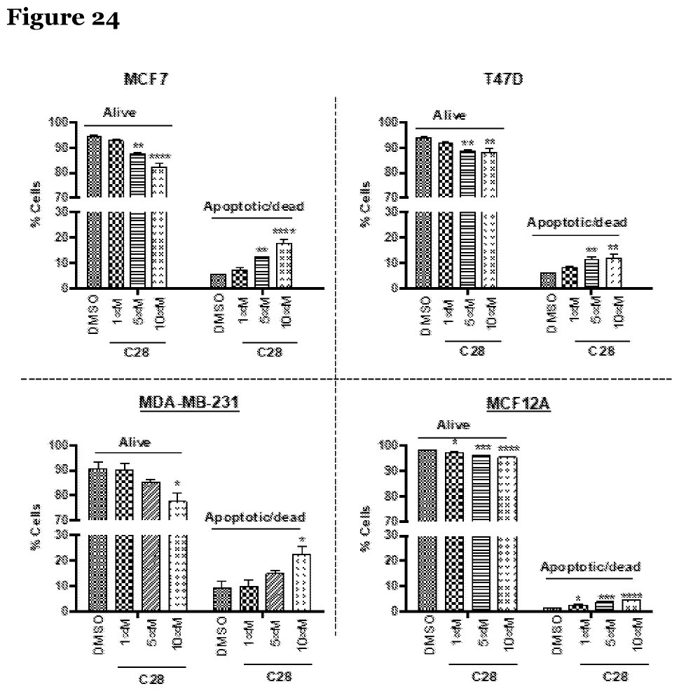

FIG. 24 MCF7, T47D, MDA-MB-231 and MCF12A cell lines were treated with increasing concentrations (0, 1, 5, 10 μM) of C28 for 72 h and the percentage of apoptotic and dead cells were analyzed by Annexin-V and 7-AAD staining. Results are expressed as mean±SEM; * P<0.05, ** P<0.01, *** P<0.001, **** P<0.0001. Each experiment has been conducted at least three times;

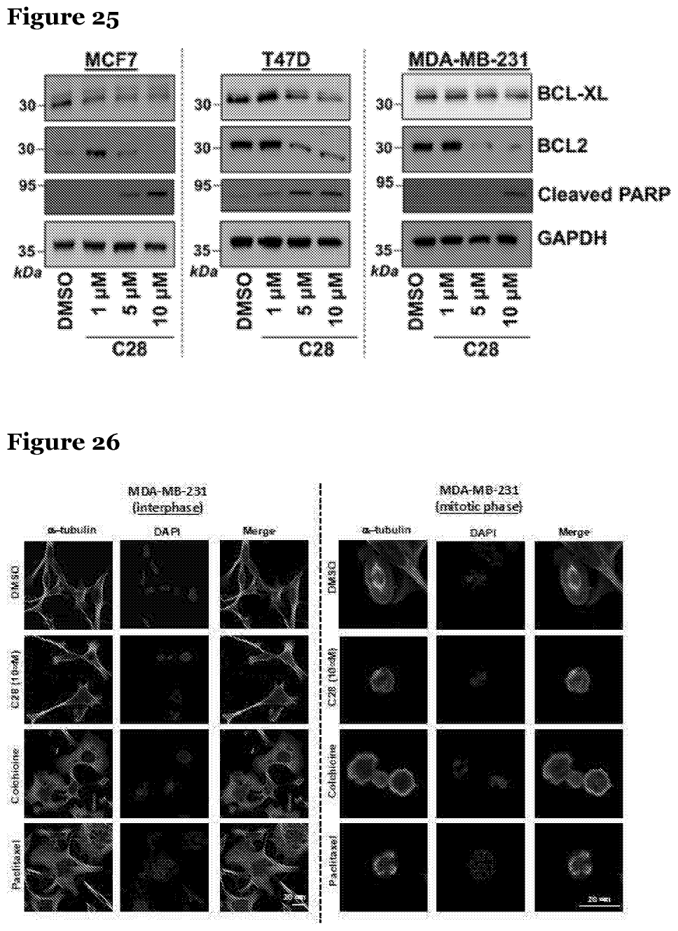

FIG. 25 shows Western blotting analysis of anti-apoptotic proteins and cleaved PARP levels in MCF7, T47D, MDA-MB-231 and MCF12A cell lines following treatment with increasing concentrations of C28 for 72 h. GADPH was used as loading control;

FIG. 26 shows representative confocal microscopy images of interphase (left panel) and mitotic phase MDA-MB-231 cells (right panel) cells e treated with 10 μM of C28 for 48 h. Cells were fixed and stained with α-tubulin antibody (green) while the nuclear

DNA was stained by DAPI (blue). Colchicine (50 nM) or Paclitaxel (50 nM) served as positive or negative controls respectively. Scale bar, 20 μm. Arrows indicate disorganized microtubule distribution in interphase cells;

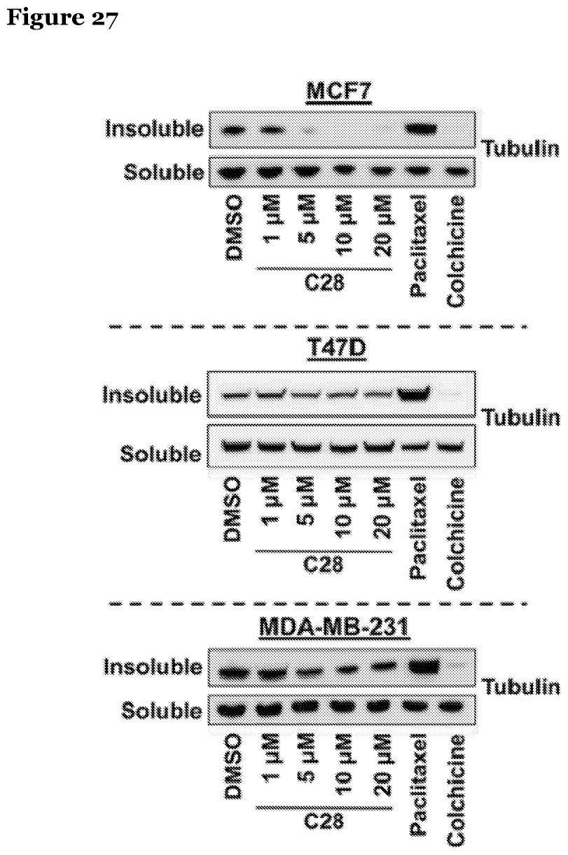

FIG. 27 MCF7, T47D and MDA-MB-231 cell lines were treated with increasing concentrations (0, 1, 5, 10 and 20 μM) of C28 for 48 h. Cells were centrifuged to separate the insoluble (polymerized) and soluble (un-polymerized) tubulin, and fractions were analyzed by western blotting. Colchicine (50 nM) or Paclitaxel (50 nM) served as positive or negative control respectively;

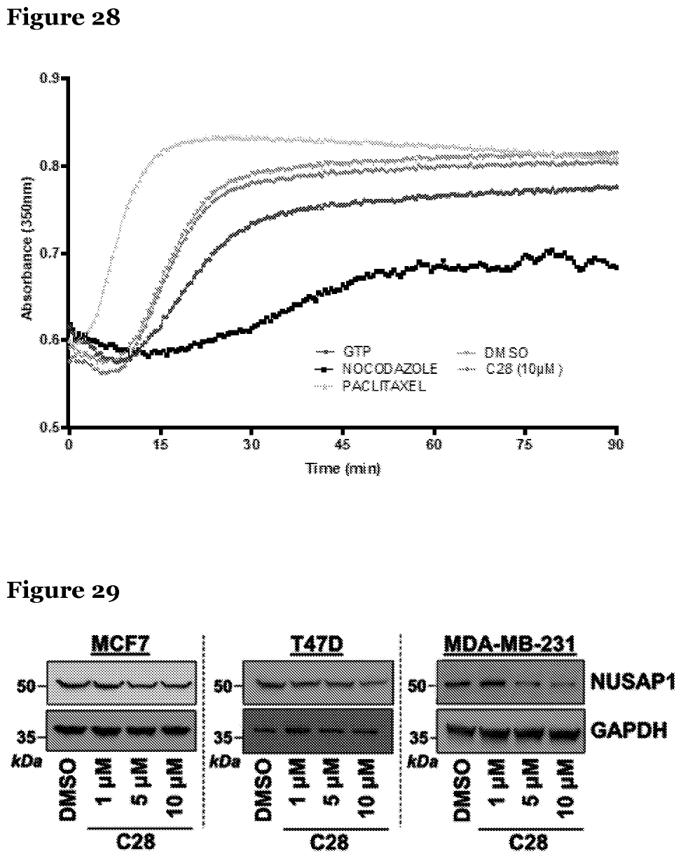

FIG. 28 In vitro polymerization of bovine purified tubulin following incubation with increasing concentrations (0, 1, 5, 10 and 20 μM) of C28. Nocodazole (10 μM) or Paclitaxel (10 μM) served as positive or negative controls respectively. The optical density (OD) was measured at 350 nm;

FIG. 29 shows Western blotting analysis of NUSAP1 in MCF7, T47D and MDA-MB-231 cell lines following treatment with increasing concentrations (0, 1, 5 and 10 μM) of C28 for 48 h. GADPH was used as loading control;

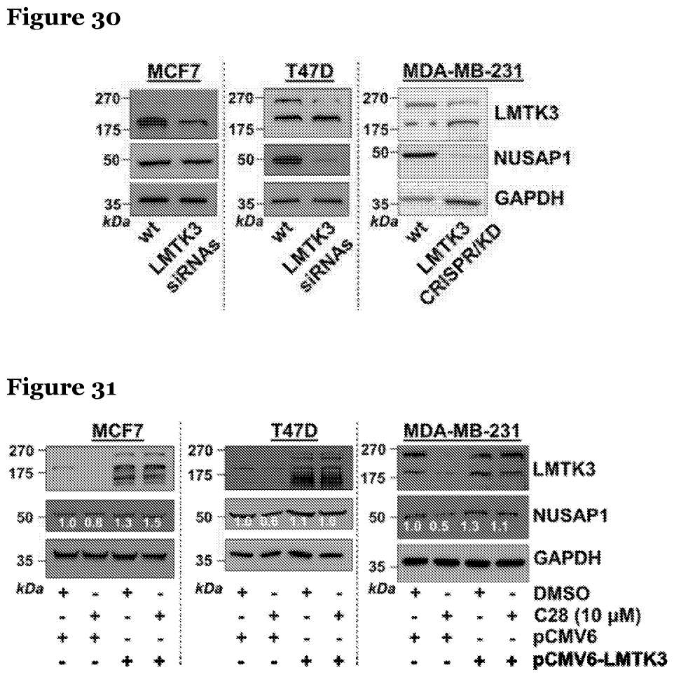

FIG. 30 shows Western blotting of NUSAP1 in MCF7, T47D and MDA-MB-231 cell lines following knock-down (siRNA) of LMTK3. GADPH was used as loading control;

FIG. 31 shows Western blotting showing the effects of LMTK3 overexpression, using pCMV6-LMTK3 plasmid, on NUSAP1 protein levels in MCF7, T47D and MDA-MB-231 cell lines following pre-treatment with 10 μM C28. GADPH was used as loading control;

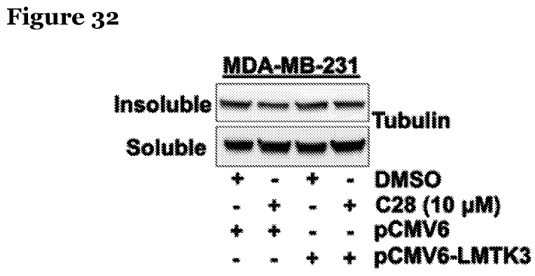

FIG. 32 shows Western blotting analysis showing the effects of LMTK3 overexpression, using pCMV6-LMTK3 plasmid, on the insoluble (polymerized) and soluble (un-polymerized) tubulin levels in MCF7, T47D and MDA-MB-231 cell lines following pre-treatment with 10 μM C28;

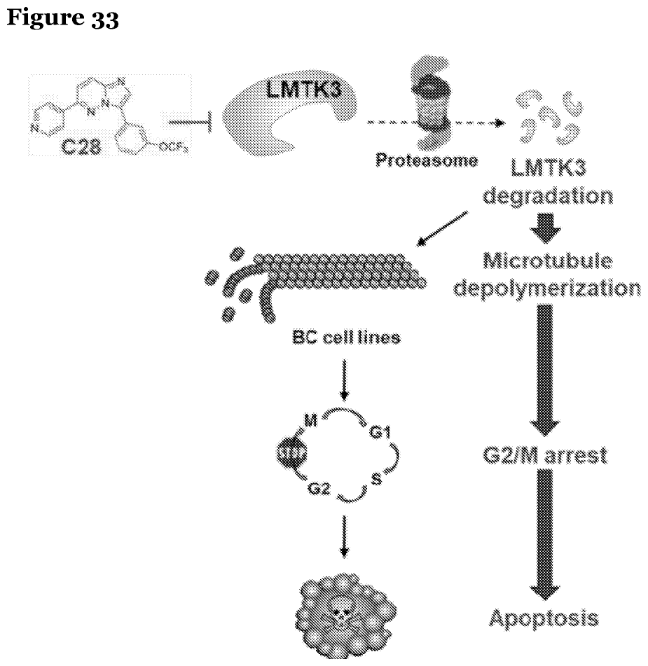

FIG. 33 is a schematic model depicting the mechanism of action of LMTK3 (C28) inhibitor. C28 ATP competitive inhibitor selectively binds to LMTK3 promoting its proteasome-mediated degradation. Downregulation of LMTK3 leads to a decrease in NUSAP1 protein levels resulting in tubulin depolymerization and a disruption in microtubules organization. Consequently, cancer cells initially arrest at G2/M phase and subsequently die;

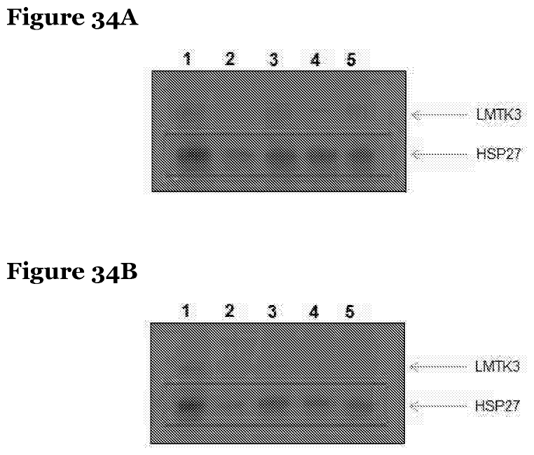

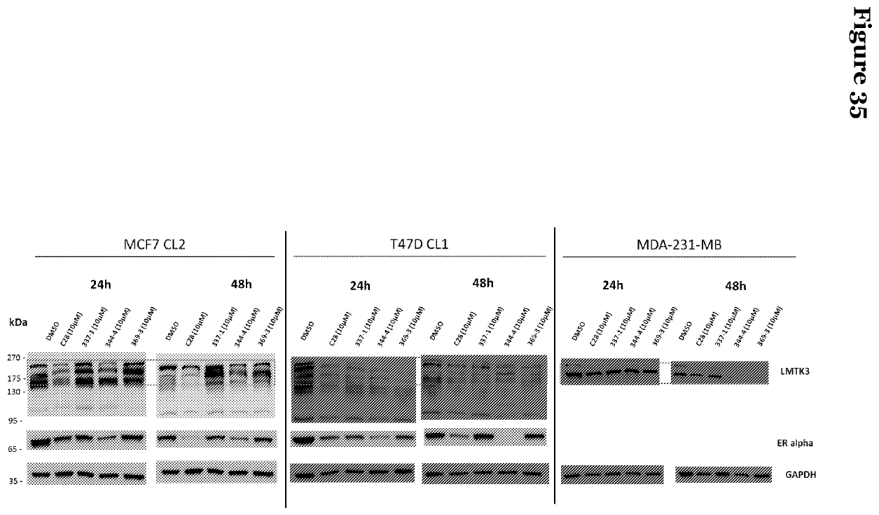

FIG. 34 shows the results of an in vitro kinase assay where 1 indicates DMSO, 2 indicates C28, 3 indicates 337-1, 3 indicates 344-5 and 5 indicates 369-3. The compounds were provided at a concentration of (A) 500 nM and (B) 1 μM;

FIG. 35 shows Western blotting analysis of LMTK3 and ERα protein levels in MCF7, T47D and MDA-MB-231 cell lines following treatment with C28 and derivatives thereof at different time points; and

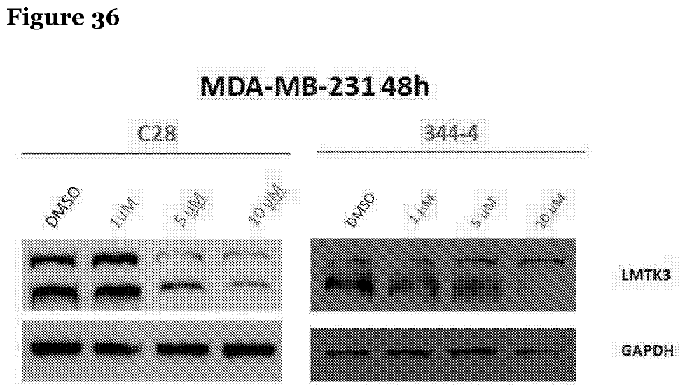

FIG. 36 shows Western blotting analysis of LMTK3 protein levels in MDA-MB-231 cell lines following treatment with C28 and 344-4 at different concentrations for 24 hours.

### EXAMPLES

The crystal structure of Lemur tyrosine kinase 3 (LMTK3) has been solved and it was determined that it possesses all the hallmarks of an active protein kinase. In particular, the K/E/D/D signature motif plays important structural and catalytic roles and comprises residues Lys193 in the β3-strand, Glu210 in the center of the αC-helix, Asp295 of the catalytic loop and Asp313, the first residue of the activation segment. Although the inventors' crystallization experiments stabilized LMTK3 in an inactive conformation, which is common in such studies, they could clearly detect the catalytic activity of LMTK3 in their biochemical assays.

In LMTK3, the activation segment DFG motif is instead DYG (residues 313-315).

Although a DYG motif is not common, it has been reported in leucine-rich repeat kinase 2 (LRRK2) associated with Parkinson's disease and atypical protein kinase C (aPKC). In the inactive “D313-out” state, Tyr314 is in an ‘in’ conformation pointing past the gatekeeper residue Met239 and occupies regions of space that would overlap with the adenine ring of bound ATP. The inventors realized that the design of kinase inhibitors that target the inactive ‘DFG-out’ conformation (type II inhibitors) offered potential of higher selectivity as compared to type I kinase inhibitors that target the active state, in addition to a profound impact on cellular activity.

### Example 1—High Throughput Homogeneous Time-Resolved Fluorescence (HTRF) Screening Identifies a Novel ATP-Competitive Inhibitor Targeting LMTK3

### Methods

Recombinant LMTK3 Protein Production

The wt kinase domain of LMTK3 (aa 134 to aa 444) was cloned into pOPINEneo which confers a C-terminal 8×Histidine tag, using InFusion™ technology (Berrow N S, et al. (2007) A versatile ligation-independent cloning method suitable for high-throughput expression screening applications. Nucleic acids research 35(6):e45). This plasmid was co-transfected into Spodoptera frugiperda (Sf9) cells with linearized Autographa califonica baculovirus bacmid (Zhao Y, Chapman D A, & Jones I M (2003) Improving baculovirus recombination. Nucleic acids research 31(2):E6-6) using Fugene HD (Promega, cat. no. E2311) (Berrow N S, et al.). Briefly, 0.5 ml of Sf9 cells were plated in a 24-well plate at 5×10⁵ cells/ml and allowed to attach. A transfection cocktail was then made by mixing 2.5 μl of linearized bacmid with 100-500 ng of plasmid DNA in 50 μl of SF900III medium (Thermo Fisher Scientific, cat. no. 12658001). To this, 1.5 μl of Fugene HD was added, mixed and the cocktail incubated at room temperature for 30 minutes. The transfection cocktail was then added to the attached Sf9 cells in the 24 well plate. The plate was incubated at 27° C. for 7 days before harvesting the supernatant which contained the P0 virus. This virus was amplified by infecting 50 ml of cells in suspension at 1×10⁶ cells/ml with 100 μl of virus. Cells were incubated at 27° C. for 7 days with shaking at 120 rpm. The P1 virus was harvested and filter sterilized before use. 2.5 L Sf9 cells at 1×10⁶ cells/ml were infected with 2.5 ml of P1 virus and incubated for 3 days at 27° C. with shaking at 120 rpm. The cells were harvested by centrifugation and frozen prior to purification.

The pellet from 2.5 L of Sf9 culture was defrosted and resuspended in ˜100 ml lysis buffer (50 mM Tris pH 7.5, 500 mM NaCl, 30 mM imidazole, 0.2% Tween 20) containing 5 μl 250 U/μl Benzonase nuclease nuclease (Sigma-Aldrich, cat. no. 9025-65-4) and 50 μl protease inhibitor cocktail (Sigma-Aldrich, cat. no. P8849). Cells were broken by passing through a Basic Z cell disruptor at 35 kPsi before removal of the cell debris by centrifugation at 30,000 g for 30 minutes at 4° C. After clarification, the cell lysate was filtered through a 0.2 μm filter before being applied to a 1 ml HisTrap FF column (GE Healthcare, cat. no. 11-0004-58). After washing with 50 mM Tris pH 7.5, 500 mM NaCl, 30 mM imidazole, the protein was eluted in 50 mM Tris pH 7.5, 500 mM NaCl, 500 mM imidazole before being automatically applied to a HiLoad 16/60 Superdex 200 column (GE Healthcare, cat. no. 8-9893-35) equilibrated in 20 mM Tris pH 7.5, 200 mM NaCl. All purification steps took place at 4° C. Fractions containing the wt LMTK3 kinase domain (wt LMTK3-KD) were analyzed by SDS-PAGE before being combined and concentrated. On average 1 mg of purified protein (>95% pure by SDS-PAGE) could be obtained per litre of infected Sf9 cells. The identity of the protein was confirmed by intact protein mass spectrometry (measured mass=36806.44 Da; expected mass with 1× acetylation=36805.13 Da) (Nettleship J E, Brown J, Groves M R, & Geerlof A (2008) Methods for protein characterization by mass spectrometry, thermal shift (ThermoFluor) assay, and multiangle or static light scattering. Methods Mol Biol 426:299-318). Similar protocol was used for generating GFP protein (negative control) as well as the mutant (kinase-dead; mut LMTK3-KD) recombinant LMTK3 protein (aa 134 to aa 444), which included the following mutations K193A, D295A and D313A.

Homogeneous Time-Resolved Fluorescence (HTRF) Chemical Compound Screening

The peptide phosphorylation activity of LMTK3 was measured using the STK Substrate 1 (S1) biotin substrate from the HTRF KinEASE kit (Cisbio Bioassays, cat. no. 62ST1PEB), according to the manufacturer's instructions. Following analysis and optimization steps, the screening assays were carried out in low volume, black 384-well plates (Corning Life Sciences, MA), with a 10 μl assay volume containing 3 μM ATP, 50 nM STK-S1 biotin and 25 ng/well of LMTK3 recombinant kinase domain (wt LMTK3-KD). After incubation at 37° C. for 2 hours, the reaction was stopped with buffered EDTA, which contained the detection reagents, streptavidin-XL665 and the STK-antibody labelled with Eu3+-cryptate. The resulting TR-FRET signal, calculated as the fluorescence ratio at 665/620 nm, was read on an Envision and was proportional to the level of phosphorylation of the peptide. A library of 28,716 kinase inhibitor-biased compounds screened against wt LMTK3-KD were initially tested at a single concentration (20 μM) and the % of inhibition of the phosphorylation of STK-S1 peptide was determined. Further hit confirmation of the top 868 compounds showing >50% mean inhibition was done (20 μM, in duplicate). Finally, potency and LC-MS purity analysis of the top 160 inhibitors (>50% inhibition) was performed (10 different concentrations, in duplicate) and the IC₅₀ values were calculated. All of the 28,716 compounds use for the screening were selected considering Lipinski's rule of five properties (175<MW<=500, AlogP<=5, ACC<=10, Don<=5, TPSA<=140).

### Results

Initially, the inventors screened a library encompassing 28,716 compounds to detect substrate phosphorylation using a homogeneous time-resolved fluorescence (HTRF) assay as a readout. Approximately ˜15% of the tested compounds displayed a >50% inhibition at 20 μM concentration. The inventors then narrowed down the hits to n=160 based on their potency, physicochemical and structural properties and performed a 10-point concentration-response profiling that gave a favorable range of IC₅₀ values (24 nM to 16 μM). Eventually, 38 out of the 160 most active compounds (IC₅₀: 24 nM-2.6 μM; see FIG. 1) were selected for follow-up in vitro and cell-based efficacy studies.

Dose-dependent in vitro ³²P γ-ATP radiolabeled kinase assays revealed variability of the inhibitory effects of the tested compounds with some of them demonstrating higher efficiency at low concentrations (<1 μM) as measured by the phosphorylation of HSP27 by LMTK3 (see FIGS. 1 and 2). The inventors then used the IL-3 dependent murine bone marrow derived cell line FDCP1 (Cleland WW (1977) Determining the chemical mechanisms of enzyme-catalyzed reactions by kinetic studies. Adv Enzymol Relat Areas Mol Biol 45:273-387) and engineered an LMTK3-transformed clone (FDCP1-LMTK3) that relies on the constitutive expression of catalytically active LMTK3 for its survival and proliferation. Using this cell-based model the inventors assessed the functional responses and potency of the selected inhibitors and determined their effective concentration (EC₅₀) values by monitoring the viability of FDCP1-parental and FDCP1-LMTK3 cell lines (see FIGS. 1 and 3).

Taking into consideration all of the results summarized in FIG. 1 and considering the novelty and physicochemical properties/similarities of the top hit compounds, the inventors focused on compound C28, i.e. 3,6-disubstituted imidazo[1,2-b]pyridazine with the following structure:

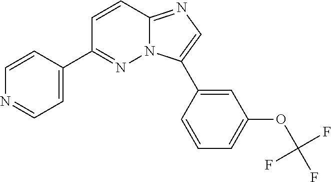 [SMILES: FC(F)(F)Oc1cccc(-c2cnc3ccc(-c4ccncc4)nn23)c1]

Other compounds to show promising results had the following structures:

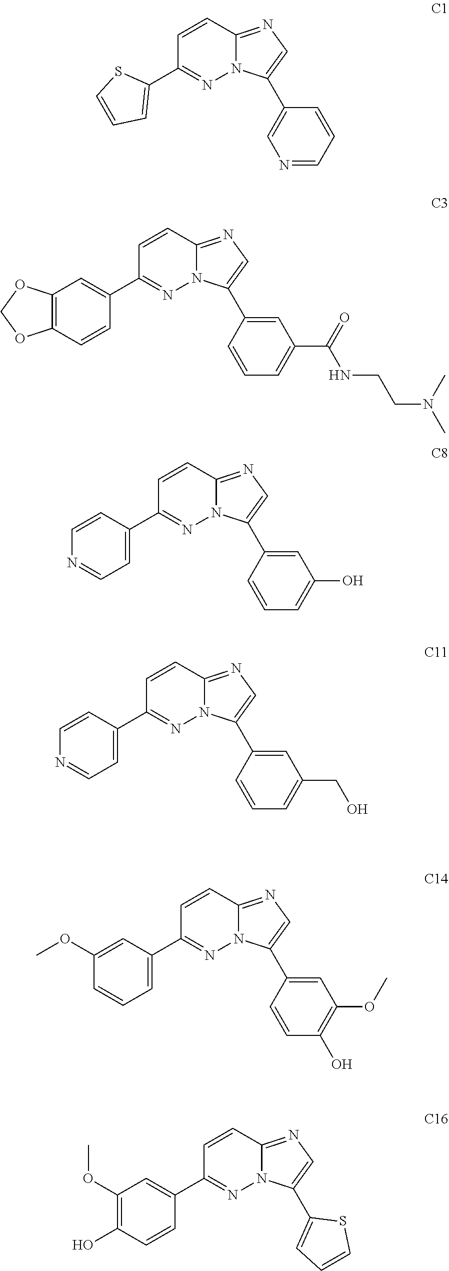

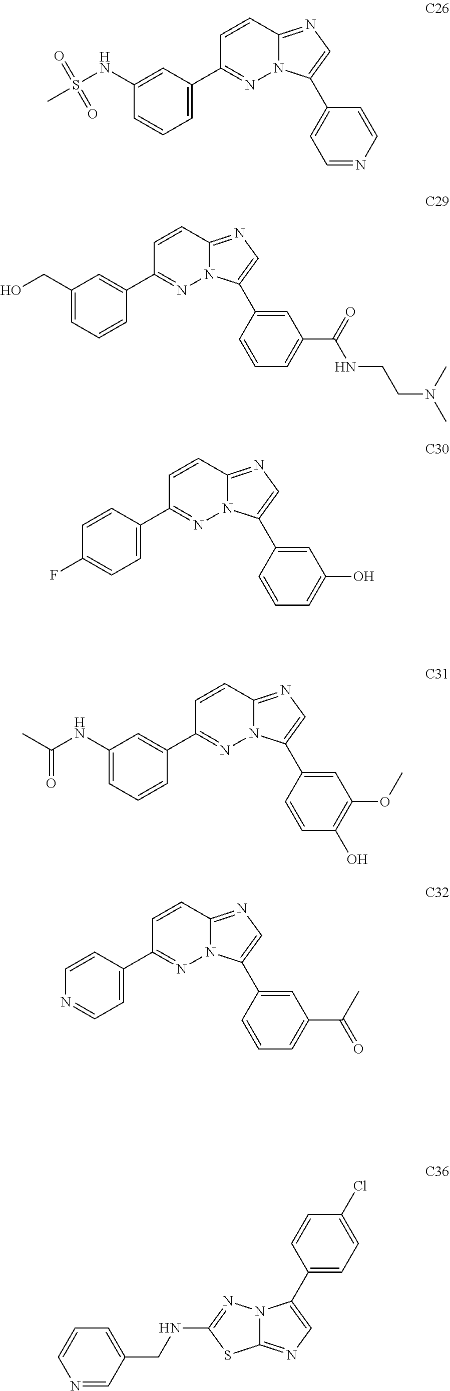

### Example 2—Further Analysis of Compound C28

### Materials and Methods

Solvents were used as purchased including deuterated solvents for NMR use. NMR spectra were recorded on a Varian NMR 600 (1H 600 MHz; 13C {1H} 151 MHz). Chemical shifts are reported in ppm. Spectra are referenced to the corresponding protic solvent (1H) or signals of the solvent (13C). The progress of reactions was monitored by thin layer chromatography (TLC) using commercially available glass silica gel plates (60 Å, F254). The mobile phase was usually a solvent mixture and the visualization was undertaken using UV light. Chromatographic purifications were carried out on an ISCO Combi Flash RF 75 or 150 PSI purification unit, gel columns. LC-MS purity analyses were undertaken using a 5 μm C18 110 Å column. % purity analysis was performed using a 30 min method in water/acetonitrile with 0.1% formic acid (5 min at 5%, 5-95% over 20 min, 5 min at 95%) with the UV set to 254 nm. High-resolution mass spectrometry (HRMS) were carried out at the University of Sussex.

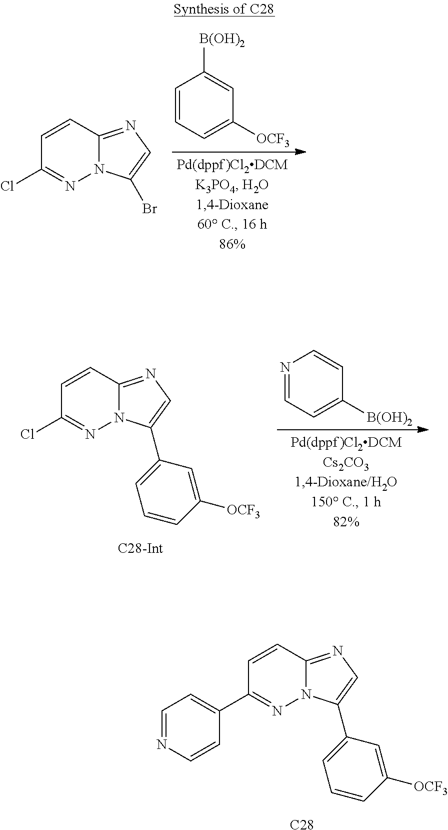

Preparation of 6-Chloro-3-(3-(trifluoromethoxy)phenyl)imidazo[1,2-b]pyridazine (C28-Int)

A mixture of 3-bromo-6-chloroimidazo[1,2-b]pyridazine (2.00 g, 8.60 mmol), potassium phosphate (5.48 g, 25.80 mmol), 3-(trifluoromethoxy)phenylboronic acid (1.86 g, 9.30 mmol), water (0.77 mL, 43.00 mmol), and anhydrous 1,4-dioxane (30 mL) was degassed under argon for 30 min. To the mixture was added [1,1′-bis(diphenylphosphino)ferrocene]dichloropalladium(II). CH₂Cl₂ (352 mg, 0.43 mmol) and the resulting mixture was heated to 60° C. for 16 h under an argon atmosphere. The resulting mixture was cooled to ambient temperature, filtered through Celite®, and concentrated under reduced pressure to give a dark orange gum (3.6 g). The resulting residue was purified by automated flash column chromatography (n-hexane/ethyl acetate, 100:0-0:100, 100 g SiO₂). The appropriate fractions were combined and concentrated under reduced pressure to give 6-chloro-3-(3-(trifluoromethoxy)phenyl)imidazo[1,2-b]pyridazine as a pale yellow solid (2.32 g, 86%). LCMS (UV, ESI) Rt=20.37 min, [M−H]+m/z=313.9, 96% purity. 1H NMR (600 MHz, d6-DMSO): δ=8.46 (1H, s), 8.34 (1H, d, J=9.4 Hz), 8.17 (1H, s), 8.15 (1H, d, J=8.0 Hz), 7.69 (1H, m), 7.48 (1H, d, J=9.5 Hz), 7.42 (1H, d, J=8.3 Hz).

Preparation of 6-(Pyridin-4-yl)-3-(3-(trifluoromethoxy)phenyl)imidazo[1,2-b]pyridazine (C28)

A mixture of 6-chloro-3-(3-(trifluoromethoxy)phenyl)imidazo[1,2-b]pyridazine (980 mg, 3.13 mmol), 4-pyridinylboronic acid (578 mg, 4.70 mmol), cesium carbonate (3.06 g, 9.39 mmol), anhydrous 1,4-dioxane (15 mL), and water (5 mL) was degassed under argon for 30 min. To the mixture was added [1,1′-bis(diphenylphosphino)ferrocene]dichloropalladium(II). CH₂Cl₂ (128 mg, 0.157 mmol) and the resulting mixture was heated to 150° C. under microwave irradiation for 1 h. To the resulting mixture was added ethyl acetate (20 mL) and water (20 mL). The resulting biphasic mixture was separated and the aqueous phase extracted with ethyl acetate (3×20 mL). To the combined organic extracts was added 2M aqueous HCl (10 mL) and the resulting mixture separated. The resulting organic phase was extracted with 2M aqueous HCl (10 mL). The combined aqueous extracted were basified by the slow addition of saturated aqueous NaHCO₃. To the mixture was added ethyl acetate (25 mL) and the resulting biphasic mixture separated. The aqueous phase was extracted with ethyl acetate (2×25 mL) and the combined organic extracts washed with brine (50 mL), dried over anhydrous MgSO₄, filtered, and concentrated under reduced pressure to give a dark yellow solid (1.3 g). The resulting residue was purified by automated column chromatography (ethyl acetate/methanol, 100:0-75:25, 45 g SiO₂) followed by reverse phase chromatography (water (+0.1% formic acid)/acetonitrile (+0.1% formic acid), 95:5-5:95, 30 g C18). The appropriate fractions were combined and concentrated under reduced pressure to give 6-(pyridin-4-yl)-3-(3-(trifluoromethoxy)phenyl)imidazo[1,2-b]pyridazine (C28) as a bright yellow solid (910 mg, 82%). LCMS (UV, ESI) Rt=14.46 min, [M−H]+m/z=356.9, 100% purity. 1H NMR (600 MHz, d6-DMSO): δ=8.81 (2H, d, J=5.7 Hz), 8.50 (1H, s), 8.43 (1H, d, J=9.5 Hz), 8.36 (1H, s), 8.25 (1H, s), 8.11-8.08 (2H, m), 8.03 (1H, d, J=9.5 Hz), 7.71 (1H, m), 7.42 (1H, m). 13C NMR (151 MHz, d6-DMSO): δ=150.6, 149.2, 148.7, 142.3, 139.9, 135.0, 130.9, 130.4, 126.9, 125.2, 121.0, 120.2, 118.2, 116.1 (one carbon not observed). HRMS (ESI-[+H]) m/z: Calcd for C₁₈H₁₂F₃N₄O 357.0958; Found 357.0953.

Differential Scanning Fluorimetry

Differential scanning fluorimetry was performed using Roche Light Cycler 96 RT-PCR instrument with excitation and emission wavelengths set to 533 and 572 nm, respectively. Solutions comprising 16 μl of 5.4 μM LMTK3 in 200 mM Tris buffer (pH 8.0), 200 mM NaCl and 4 μl of 50× SYPRO orange (Sigma) and 0.2 μl of either DMSO or C28 in DMSO (final concentration of 10 μM C28, 1% v/v DMSO, 4.3 μM LMTK3 and 10× SYPRO orange). The temperature range spanned 25 to 80° C. at a scan rate of 1° C./min. Data analysis was performed in LightCycler 96 (v1.1) software using the melting curve analysis and Tm values were determined as the first negative derivative of the fluorescence with respect to the temperature.

Circular Dichroism Spectroscopy

Circular dichroism spectroscopy was performed using a Jasco J instrument. Temperature was increased from 20° C. to 90° C. at an increment of 1° C./min and data points were acquired every 0.2° C. by monitoring a wavelength of 230 nm. For thermal stability experiments, LMTK3 samples of 5.4 μM in 200 mM Tris buffer (pH 8.0), 200 mM NaCl were treated with either DMSO 0.4% (v/v) or 8.3 μM C28 in DMSO (0.4%) to a total volume of 120 μl in 0.1 cm curvets. Data analysis was performed in GraphPad Prism (v7.0) by fitting data in the transition region to a Boltzmann sigmoidal. Apparent Tm values were determined as the point in which the transition was 50% complete.

### Results

The inventors assessed the ability of C28 to bind to LMTK3 by monitoring the thermal denaturation of LMTK3 and the LMTK3/C28 complex using differential scanning fluorimetry (DSF) and circular dichroism (CD) spectroscopy. As shown in FIG. 4, both methods revealed an increase of the thermal stability of LMTK3 in the presence of C28, characteristic of ligand binding. The increase in the Tₘ value of LMTK3 upon C28 binding was determined at 2.6° C. using DSF (50.4→53.0° C.; P=0.001) and 3.1° C. using CD (51.14→54.2° C.; P=0.002). Only a single transition was observed in the thermal melting curves and thermal unfolding was irreversible due to protein aggregation.

To investigate the mechanism of action of C28, the inventors examined the effect of increasing substrate concentrations of HSP27 on the inhibitory activity of the compound in the presence of a constant amount of ATP. The inventors' results revealed that the inhibitor does not affect the Kₘ value (0.239 μM in the absence versus 0.249 μM in the presence of C28), but results in a significantly lower Vₘₐₓ (108.6 μmol/min versus 41.44 μmol/min) in the presence of C28 (see FIG. 5). The unchanged Kₘ value with increasing concentrations of HSP27 indicates that the inhibition is substrate independent.

The inventors next examined the effect of increasing concentrations of ATP at a fixed substrate (HSP27) concentration (0.6 μM). The data from the steady state analysis were fitted to the Michaelis-Menten equation (Cleland WW (1977) Determining the chemical mechanisms of enzyme-catalyzed reactions by kinetic studies. Adv Enzymol Relat Areas Mol Biol 45:273-387) and, as shown in FIG. 6, in the presence of C28 the inventors observed a significant increase in the apparent Kₘ value from 0.0053 μM to 0.038 μM indicating that C28 is a competitive inhibitor.

In summary, the inventors provide evidence of a novel ATP-competitive inhibitor against LMTK3.

### Example 3—C28 is a Highly Selective LMTK3 Inhibitor That Promotes Proteasome-Mediated Degradation of LMTK3

### Materials and Methods

Cell Lines

MCF7, T47D and MDA-MB-231 cell lines were purchased from ATCC. MCF7 and MDA-MB-231 cells were maintained in low glucose DMEM (Sigma Aldrich, cat. no. D6046-500ML) supplemented with 10% FBS (Sigma Aldrich, cat. no. F7524-500ML) and 1% Penicillin/Streptomycin (Sigma-Aldrich, cat. no. P0781-100ML). T47D cells were maintained in RPMI-1640 medium (Sigma Aldrich, cat. no. R5886-500ML) supplemented with 10% FBS (Sigma Aldrich, cat. no. F7524-500ML) and 1% L-glutamine/Penicillin/Streptomycin solution (Sigma-Aldrich, cat. no. G1146-100ML). MCF7/LMTK3 cell line stably overexpressing LMTK3 has been described before (Jacob J, et al. (2016) LMTK3 escapes tumour suppressor miRNAs via sequestration of DDX5. Cancer Lett 372(1):137-146). All of the cells were incubated at 37° C. with 5% CO₂.

Protein Kinase Assays

³²P γ-ATP in vitro kinase assays were performed in-house as have previously described (Perkin Elmer, cat. no. BLU002A500U, UK) (Giamas G, et al. (2009) CK1delta modulates the transcriptional activity of ERalpha via AIB1 in an estrogen-dependent manner and regulates ERalpha-AIBi interactions. Nucleic acids research 37(9)3110-3123 and Giamas G, et al. (2007) Phosphorylation of CK1delta: identification of Ser370 as the major phosphorylation site targeted by PKA in vitro and in vivo. Biochem J 406(3):389-398). The selectivity ‘premier screening’ of C28 against a panel of 140 protein kinases was performed at the MRC International Centre for Kinase Profiling unit (http://www.kinase-screen.mrc.ac.uk/services/premier-screen).

Kinase Inhibitor Competition Binding Assay

The selectivity profiling of C28 kinase inhibitor at 5 μM was analyzed using DiscoveRx KINOMEscan competition binding assay against a panel of 456 kinases (www.discoverx.com).

Western Blotting

Protein lysates were extracted using RIPA buffer (Sigma Aldrich, cat. no. R0278-50ML) including fresh protease and phosphatase inhibitors (Roche Diagnostics GmbH, cat. no. 11697498001 & cat. no. 4906845001) and standard western blotting protocol was performed as described before (Giamas G, et al. (2007) Phosphorylation of CK1delta: identification of Ser370 as the major phosphorylation site targeted by PKA in vitro and in vivo. Biochem J 406(3)389-398). Briefly, protein concentration of the lysates was determined using the Pierce BCA protein assay kit (ThermoFisher Scientific, cat. no. 23227) and 30/50 μg of protein extract was resolved on SDS/PAGE gels (GenScript, cat. no. M42012 & cat. no. M00653). Proteins were then transferred onto a nitrocellulose blotting membrane (Thermo Fisher Scientific, cat. no. IB23001) using the iBlot 2 dry blotting system (Thermo Fisher Scientific, cat. no. IB21001). The membranes were blocked in TBS containing 0.1% (v/v) Tween 20 and 5% (w/v) non-fat milk or 5% (w/v) BSA (VWR Life Science, cat. no. 421501J) for 1 hour before being incubated with the primary antibodies overnight at 4° C. HRP-conjugated secondary anti-rabbit (1:5000, Cell Signaling, cat. no. 7074P2) or anti-mouse (1:5000, Cell Signaling, cat. no. 7076P2) antibodies were used. Chemiluminescent detection was performed using the SuperSignal West Pico PLUS Chemiluminescent Substrate (Thermo Fisher Scientific, cat. no. 34577). Emission was captured using the UVP

ChemStudio Imaging Systems (Analityk jena).

### Results

To determine a more detailed profile of the selectivity of C28, the inventors screened it against a series of 140 kinases (http://www.kinase-screen.mrc.ac.uk/services/premier-screen) by performing a radioactive filter binding assay using ^{33P}γ-ATP as described (Bain J, et al. (2007) The selectivity of protein kinase inhibitors: a further update. Biochem J 408(3):297-315 and Hastie C J, McLauchlan H J, & Cohen P (2006) Assay of protein kinases using radiolabeled ATP: a protocol. Nature protocols 1(2):968-971). Interestingly, the inventors identified only 18/140 kinases whose activity was reduced >50% in the presence of 1 μM C28 out of which only 4 (CLK: 5 nM; DYRK1α: 6 nM; HIPK2: 48 nM and IRAK4: 41 nM) had similarly low IC₅₀ values to that of LMTK3 (67 nM) (FIG. 7).

Following, to further examine the specificity of C28 the inventors used an active site-directed competition binding assay (DiscoveRx KINOMEscan) and quantitatively measured the interactions between C28 and 403 purified human kinases (Fabian M A, et al. (2005) A small molecule-kinase interaction map for clinical kinase inhibitors. Nat Biotechnol 23(3):329-336 and Karaman M W, et al. (2008) A quantitative analysis of kinase inhibitor selectivity. Nat Biotechnol 26(1):127-132) (FIG. 8). The S(35) selectivity index of C28 was 0.186, as measured by the percentage of the kinome inhibited below 35% of the control at this concentration (S[35]=[number of kinases with % Ctrl<35]/[number of kinases tested]). Intriguingly, quantitative analysis of 71 kinase inhibitors currently used in clinical oncology (Karaman M W, et al. (2008) A quantitative analysis of kinase inhibitor selectivity. Nat Biotechnol 26(1):127-132) showed a comparably low S(35) score as C28. C28 inhibited by >90% the activity of only 33/403 kinases, most of which (20/33) were different isoforms of the same proteins, highlighting an overall low promiscuity of C28. It is worth mentioning that despite the different principles of the two assays employed to examine the selectivity of the inhibitor (active site-directed competition binding assay and radioactive filter binding assay), there was considerable overlap of identified kinases targeted by C28 highlighted in FIGS. 7 and 8 further validating the accuracy of the results.

Treatment of different BC cell lines with increasing concentrations of C28 resulted in time and dose-dependent degradation of LMTK3 (FIG. 9), which was further validated following treatment with cyclohexamide, as shown by the decrease in half-life of LMTK3 in the presence of C28 (FIG. 10). Consequently, the inventors then analyzed the effects of C28 on LMTK3 protein turnover following co-treatment with the proteasomal inhibitor MG132 and observed that MG132 is able to prevent C28-induced downregulation of LMTK3 (FIG. 11). These results suggest that C28 promotes proteasomal degradation of LMTK3, also in turn associated with increased LMTK3 ubiquitination (FIG. 12).

The inventors have previously reported that silencing of LMTK3 results in downregulation of estrogen receptor alpha (ERα) and indeed, treatment with C28 decreased protein levels of ERα (FIG. 9). Moreover, the inventors also observed a reduction in the phosphorylation state of HSP27 in BC cells suggesting that HSP27 is also an in vivo (apart from in vitro) substrate for LMTK3. Combined, these results further supported the specificity of C28 inhibitor against LMTK3 (FIG. 13). Finally, the inventors also examined the effects of C28 on CLK2, DYRK1α, HIPK2, IRAK4 and TRKA kinases (total levels and phosphor-sites linked to their activities), which were identified as potential C28 targets from the selectivity screening. As shown in FIG. 14, treatment of BC cell lines with 10 μM of C28 for different time points led to degradation of IRAK4 and HIPK2.

In aggregate, this is the first report identifying a highly selective LMTK3 inhibitor, which appears to have a secondary selectivity towards IRAK4 and HIPK2.

### Example 4—The C28 Inhibitor Exhibits Potent Anticancer Activity in Different Human Cancer Cell Lines and Inhibits Tumor Growth in Mouse Models

### Materials and Methods

Cell Lines

MCF12A cell lines were purchased from ATCC. MCF12A cells were maintained in Cascade Biologics medium 171 (Gibco, cat. no. 171 M-171-500) supplemented with 10% FBS (Sigma Aldrich, cat. no. F7524-500ML), 1% L-Glutamine/Penicillin/Streptomycin (Sigma Aldrich, cat. no. G1146-100ML), 1% mammary epithelial growth supplement (Thermo Fisher Scientific, cat. no. S0155) and 100 ng/ml cholera toxin from Vibrio cholerae (Sigma Aldrich, cat. no. C8052-5MG).

The other cell lines were as described in Example 3. All of the cells were incubated at 37° C. with 5% CO₂.

Animal Experiments

For xenograft generation, MDA-MB-231 cells (1×10⁶) were injected subcutaneously into the left and right flank of 4-5 weeks old NSG mice (NOD-scidIL2Rgammanull, The Jackson Laboratory). Once tumors were palpable (approximately 0.4-0.5 cm in diameter) mice started a 3-week treatment by oral gavage daily, with one break per week. The treatment groups received either vehicle (5% dextrose/PEG400 in 1:1 ratio) or C28. To prepare the formulation, C28 was first dissolved in the necessary volume of PEG400, vortexed for 1 minute, and then sonicated for 30 minutes at 40° C. Next, 5% dextrose solution in water was added to the formulation under vigorous vortexing. Tumors were monitored twice weekly by caliper measurements. In total, 3 mice died during the treatment (C28-treated animals).

For the MDA-MB-231-Luc xenograft experiment, 2×10⁶ MDA-MB-231-Luc cells were suspended in 50 μl 1× PBS and 50 μl Matrigel (1:1 ratio) and injected subcutaneously into the flanks of 5 weeks old female NU/J homozygous mice (Jackson laboratory, Maine, USA). MDA-MB-231-Luc cells (before injection) and the tumor implanted mice were imaged immediately after cell injection using D-luciferin (Promega, WI, USA) dissolved in PBS and injected intraperitoneal at a dose of 1.5 mg per mouse, 15 min prior to measuring luminescence using IVIS 100 Bioluminescence/optical imaging system (Xenogen Corporation, CA, USA). Mice were monitored daily for any discomfort, tumor growth and body weight. Once the tumors reached approximately 1 cm size, mice were randomly divided into 3 groups: (i) vehicle: 5% dextrose/PEG400 25%:75%, v/v (n=6), (ii) C28: 10 mg/kg (n=6) and (iii) C28: 30 mg/kg (n=5). The animals were imaged before the treatment started (days 0 of the treatment) to measure the tumor growth. Treatment was given for 21 days daily through oral gavage. Mice were imaged on day 14 and 21 post treatment.

MMTV-Neu mice (Dankort D, et al. (2001) Grb2 and She adapter proteins play distinct roles in Neu (ErbB-2)-induced mammary tumorigenesis: implications for human breast cancer. Molecular and cellular biology 21(5):1540-1551) were treated by oral gavage daily for a total of 19 days, as soon as tumors were palpable. The treatment groups received either vehicle (5% dextrose/PEG400 in 1:1 ratio) or C28. Tumors were monitored twice weekly by caliper measurements. Tumor volumes were calculated using the following formula: V=a×b2/2, where “a” is the largest diameter and “b” is the smallest. All animal studies were performed in accordance with national and international regulations and were approved by the BRFAA ethical committee.

### Results

The inventors then investigated the potential use of C28 as an anticancer strategy by examining the proliferation of various BC cell lines (and one non-transformed breast cell line; MCF12A) in the presence of increasing concentrations of C28. As shown in FIG. 15, C28 was able to inhibit the growth of BC cells with IC₅₀ values ranging from 6.5-8.64 μM, while the non-cancerous cell line (MCF12A) appeared to be less sensitive to C28 (IC₅₀:14.77 μM).

The inventors next submitted C28 to the Developmental Therapeutic Program (DTP) of the National Cancer Institute (NCI) and screened it against a panel of 60 human cancer cell lines (Shoemaker R H (2006) The NCI60 human tumour cell line anticancer drug screen. Nature reviews. Cancer 6(10):813-823). Remarkably, at a 10 μM dose, C28 inhibited all of the cancer cell lines by >50% (FIG. 16). Considering the significant growth inhibition demonstrated, the NCI decided to further evaluate C28 at five doses.

The results revealed that most of the leukemia, melanoma, ovarian and CNS cell lines were more resistant to C28, while renal cancer cells appeared to be more sensitive. These data demonstrate possible anticancer applicability of C28 in different tumor types.

Before testing the utility of C28 in mouse models, its solubility was assessed in various formulation vehicles and dissolution conditions. The inventors also investigated the pharmacokinetic (PK) properties of C28 in nude Balb/c mice at different doses following intravenous (IV), intraperitoneal (IP) and oral (PO) administration. Analysis of plasma samples by LC-MS/MS revealed that IV-dosed C28 (5 mg/kg) had a peak concentration (Cₘₐₓ) of 1533 ng/ml at 5 min after injection, which was countered by a short half-life (T_{1/2}˜10 min), whereas lowering the dose to 1 mg/kg led to very little exposure (Cₘₐₓ=185 ng/ml). The optimal route of administration appeared to be IP at 10 mg/kg, where Cₘₐₓ was 839 ng/ml wth a T_{1/2} of ˜59 min) or PO (Cₘₐₓ=632 ng/ml; T_{1/2}˜62 min), see FIG. 17.

As the inventors showed PO bioavailability of C28, they evaluated its antitumor activity in the MMTV-Neu mammary tumor model transgenic model in which a constitutively active mouse Neu protein is expressed under control of the mouse mammary tumor virus promoter. Once tumors became palpable, mice were treated PO daily, with either control vehicle (5% dextrose/PEG400 in 1:1 ratio) or C28 at a final concentration of 10 mg/kg. As shown in FIG. 18, C28 nearly completely abrogated tumor growth compared to the vehicle-treated group. Histopathologic analysis of tumor sections and other organs (lung, stomach, heart, kidney, liver, spleen) between different groups did not show any significant differences, further demonstrating the lack of toxicity of the C28 inhibitor. Moreover, Ki-67 immunohistochemistry (IHC) analysis showed decreased proliferation following treatment with C28 (FIG. 19) while IHC also revealed a decrease of LMTK3 protein levels (FIG. 20), similar to our cell-based observations.

Next, the antitumor activity of the LMTK3 inhibitor was also evaluated using a BC xenograft mouse model where MDA-MB-231 TNBC cells were injected subcutaneously into immunocompromised mice. Once tumors were palpable, mice were divided in different groups and were treated PO daily at either control vehicle or C28 with a final concentration of 10 mg/kg. After 23 days, treatment with C28 significantly impeded growth compared to the control group (FIG. 19), with no changes in body weight observed (vehicle group: Day 0: 21.22 gr→Day 15: 21.42 gr vs C28-group Day 0: 21.77 gr→Day 15: 21.84 gr). Similar results were obtained in a separate study using luciferase labeled MDA-MB-231 and treatment with either 10 mg/kg or 30 mg/kg of C28 (FIG. 19).

In aggregate, the data demonstrate potent pre-clinical anticancer activity of C28 in vitro and a delay of tumor onset in vivo in mice models.

### Example 5—C28 Promotes G2/M Arrest and Apoptosis by Inhibiting Tubulin Polymerization and Disrupting Microtubule Organization

### Materials and Methods

Cell Lines

The other cell lines were as described in Examples 3 and 4.

Western Blotting

Western blotting was performed as described in Example 3.

Cell Cycle Analysis

Cells were synchronized by serum starvation and then treated with increasing concentrations of C28 for 48 hours in complete medium. After collection, the DNA was labelled with propidium iodide using the BD Cycletest Plus DNA Reagent Kit (BD Biosciences, cat. no. 340242) following the manufacturer's instructions. The BD Accuri C6 flow cytometer (BD Biosciences) was used.

Mitotic Index Assay

Cells grown on glass coverslips were fixed in 4% paraformaldehyde for 15 minutes, washed in PBS and incubated with 0.3% (v/v) Triton X-100, 3% BSA in PBS for 30 minutes at room temperature. Coverslips were then stained with anti-tubulin antibody (1:100, Genscript, cat. no. A01410-100) and DAPI (Thermo Fisher Scientific, l cat. no. S36942). The mitotic index was evaluated counting the percentage of mitosis scoring at least 1000 nuclei.

Cell Death and Apoptosis

Cells were treated with increasing concentrations of C28 for 48 or 72 hours. After collection, cells were stained with the Muse Annexin V Dead Cell Kit according to the manufacturer's protocol (Millipore, cat. no. MCH100105). Cells were then analyzed using the Muse Cell Analyzer (Millipore).

Immunofluorescence Staining Cells grown on glass coverslips were fixed in 4% paraformaldehyde for 15 minutes, washed in PBS and incubated with 0.3% (v/v) Triton X-100, 3% BSA in PBS for 30 minutes at room temperature. Coverslips were then incubated overnight at 4° C. with α-tubulin primary antibody (1:100, cat. no.A01410-100) diluted in the same buffer. Cells were washed and probed with Alexa Flour®-488 goat anti-mouse secondary antibody (Thermo Fisher Scientific, cat. no. A11029) at room temperature for 60 minutes. After washing, coverslips were mounted onto glass slides with the Slowfade Gold antifade reagent with DAPI (Thermo Fisher Scientific, cat. no. S36942). Images were acquired through a 63× oil immersion lens on a Zeiss LSM880 confocal microscope equipped with a CCD camera.

In Vivo Microtubule Assembly Assay

Separation of insoluble polymerized microtubules from soluble tubulin dimers was performed as previously described (Blagosklonny M V, et al. (1995) Taxol induction of p21WAF1 and p53 requires c-raf-1. Cancer Res 55(20):4623-4626). Briefly, cells were treated for 48 hours with increasing concentrations of C28, 50 nM Paclitaxel (Sigma Aldrich, cat. no. T1912-1MG) and 50 nM Colchicine (Sigma Aldrich, cat. no. C9754-100MG). Cells were then harvested and lysed using a hypotonic lysis buffer (20 mM Tris HCl pH 8, 1 mM MgCl₂, 2 mM EGTA, 1% NP40), supplemented with protease and phosphatase inhibitors. Supernatants containing the soluble fraction were collected after centrifugation at 13,000 rpm for 10 minutes at room temperature. The pellets were extracted using RIPA buffer (Sigma Aldrich, cat. no. R0278-50ML) including fresh protease and phosphatase inhibitors (Roche Diagnostics GmbH, cat. no. 11697498001 & cat. no. 4906845001). Supernatants and pellets were dissolved in SDS-PAGE sampling loading buffer at 95° C. for 10 minutes and subjected to electrophoresis on SDS-polyacrylamide gels before Western blotting. The relative amounts of tubulin were detected by an anti-a-tubulin primary antibody (0.5 μg/ml, Genscript, cat. no. A01410-100) and horseradish peroxidase-conjugated secondary antibody. Chemiluminescent detection was performed using the SuperSignal West Pico PLUS Chemiluminescent Substrate (Thermo Fisher Scientific, cat. no. 34577). Emission was captured using the UVP ChemStudio Imaging Systems (Analityk jena).

In Vitro Microtubule Polymerization Assay

The polymerization of purified tubulin was detected after the incubation with increasing concentrations of C28 using the in vitro tubulin polymerization assay kit (Millipore, cat. no. 17-10194-1) according to the manufacturer's instructions. DMSO was used as vehicle control. Nocodazole (10 μM) and Paclitaxel (10 μM) were used as positive tubulin-targeting agents controls. The tubulin polymerization was measured by continuous monitoring of the turbidity change at 350 nm using the SpectraMax i3x plate reader.

LMTK3 Silencing

Cells were transfected with a pool of 3 LMTK3 siRNAs (s41588, s41589, s415890 Ambion) or scramble control (4390843, Ambion) using the 4D-Nucleofector™ System (LONZA), following the manufacturer's instructions. Briefly, 500,000 cells were resuspended in 20 μl of complete buffer SE (Lonza, cat. no. PBC1-00675) and 300 nM siRNA were added prior electroporation. Cells were then seeded in warm complete medium. Silencing was confirmed by western blotting.

LMTK3 Overexpression

300,000 cells were seeded per well in a 6-well plate. Cells were then transfected with 2 μg of pCMV6-LMTK3 plasmid (Origene, cat. no. RC223140) or pCMV6 empty vector (Origene, cat. no. PS100001) using Fugene HD transfection reagent (Promega, cat. no. E2311), following the manufacturer's instructions. Overexpression was then confirmed by western blotting.

### Results

In order to decipher the mechanism of action of C28, the inventors performed flow cytometry (FACS) analysis and revealed that treatment with C28 leads to G2/M phase arrest of BC cells (FIG. 21). This result was confirmed by evaluating the increased mitotic index (FIG. 22) and by the high expression of the mitotic marker phospho-Histone H3-Serio (FIG. 23). After prolonged exposure to C28, concomitant induction of apoptosis was observed (FIG. 24) with no effects on cellular senescence (data not shown).

Considering a documented link between mitotic arrest and induction of apoptosis, the inventors we examined the expression of different pro- and anti-apoptotic proteins. As expected and consistent with a cell-death apoptotic mechanism, the inventors observed a decrease in BCL-XL and BCL2 anti-apoptotic proteins and an increase in cleaved PARP in the BC cell lines (FIG. 25). Interestingly, the non-transformed MCF12A breast cell line, showed a low % of cell death following G2/M arrest (<5%) even at high concentrations of C28 and longer time points (FIG. 24) suggesting that non-transformed breast epithelial cells evade death while working on recuperating from cell cycle arrest (FIG. 21). Taken together, these results suggest that C28 displays its effects selectively against cancer cells, results that require clinical exploration.

Bearing in mind the effects of C28 on cell cycle arrest and the induction of cell death, the inventors investigated the possibility that C28 impacts tubulin polymerization, which can in turn disturb the organization of the cytoskeleton and affect cell division. Immunofluorescence of cells at the interphase and metaphase revealed that C28-treated cells present disrupted microtubule distribution, mitotic defects including abnormal microtubule spindle organization and an altered chromosome condensation pattern (FIG. 26). Cells treated with colchicine and paclitaxel, two known drugs targeting microtubules dynamics were used as controls.

The inventors confirmed the effects of C28 on microtubule dynamics using a cell-based microtubule polymerization assay where they observed a dose dependent decrease of the insoluble polymerized tubulin fraction following treatment (FIG. 27). Considering the documented role of kinase inhibitors acting on microtubules, the inventors investigated the possibility that C28 is a direct tubulin-targeting agent by employing an in vitro tubulin polymerization assay using paclitaxel and nocodazole (another well-described microtubule-destabilizing agent) as controls. Increased concentrations of C28 had no effects on tubulin polymerization, (FIG. 28) implying that C28 confers its effects by modulating LMTK3-regulated pathways linked to microtubule assembly.

The role of several proteins implicated in microtubule organization has been extensively investigated. Amongst them, nucleolar and spindle associated protein 1 (NUSAP1; also known as ANKT) is a potent microtubule-associated protein (MAP), which has been reported crucial for spindle assembly by promoting stabilization and crosslinking of microtubules adjacent to chromatin. Moreover, the involvement of NuSAP1 in cancer has been well-documented whilst its overexpression has been observed in metastatic BC and associated with a poor prognosis. Therefore, the inventors investigated whether C28 can affect NuSAP1 and observed a decrease in its protein levels following C28 treatment of different BC cell lines (FIG. 29). Similarly, after silencing of LMTK3 (siRNA) in BC cell lines, the NUSAP1 protein was downregulated (FIG. 30). To further establish that this was an LMTK3-mediated effect, the inventors restored LMTK3 levels by overexpressing LMTK3 after C28 treatment and observed a rescue of NUSAP1 levels (FIG. 31) along with a partial recovery of tubulin polymerization (FIG. 32). Finally, the inventors investigated the association between mRNA expression levels of LMTK3 and NUSAP1 with survival. Their analysis revealed that high expression of LMTK3 and NUSAP1 correlates with worse overall survival and disease free survival, suggesting a cooperation of LMTK3 and NUSAP1 in BC progression.

In summary, the inventors have demonstrated that C28 exerts its antitumor effects by degrading LMTK3, which leads to microtubule depolymerization with subsequent cell-cycle arrest and cell death (FIG. 33).

### Example 6—Derivatives of C28

### Materials and Methods

The inventors cynthesised the following compounds using the same method described in example 2 for C28, except 2-pyridine boronic acid, 3-pyridine boronic acid or 2,5-dimethyl-4-pyridine boronic acid were used instead of 4-pyridine boronic acid,

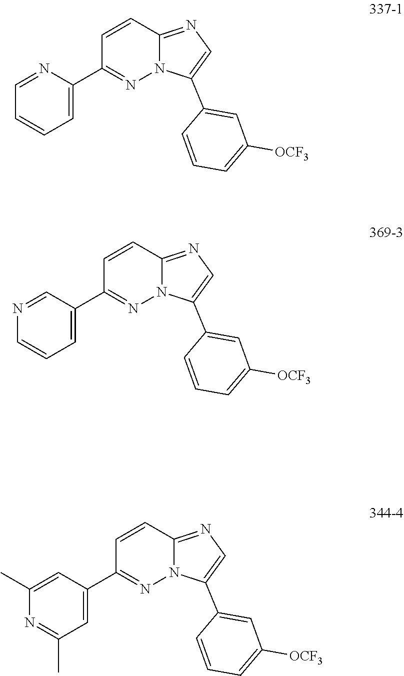 [SMILES: FC(F)(F)Oc1cccc(-c2cnc3ccc(-c4ccccn4)nn23)c1 ; FC(F)(F)Oc1cccc(-c2cnc3ccc(-c4cccnc4)nn23)c1 ; Cc1cc(-c2ccc3ncc(-c4cccc(OC(F)(F)F)c4)n3n2)cc(C)n1]

Biological Activity

The biological activity of the derivatives was evaluated using the methods described in Examples 1 to 5.

### Results

As can be seen in FIG. 34, an in vitro kinase assay indicated that C28 was a better inhibitor than any of the derivatives. However, out of the three derivatives tested, 344-4 was the best inhibitor. The approximate IC₅₀ values are given in table 1 below.

TABLE 1
Approximate IC₅₀ values for C28 derivatives

| Compound | Approximate IC₅₀ (nM) |
|---|---|
| 337-1 | 400 |
| 369-3 | 300 |
| 344-4 | 350 |

Similarly, FIGS. 35 and 36 show that C28 was also the best inhibitor when tested in cancer cell lines. However, all of the derivatives showed some activity with 344-4 being the best inhibitor of both LMTK3 and ERα.

Finally, the inventors conducted a mouse microsomal stability assay using compound 344-4. This compound demonstrated medium clearance.

### Example 7—Further Derivatives of C28

### Materials and Methods

The inventors synthesized the further derivatives of C28 using methods analogous to those given in example 2.

The ability of these compounds to inhibit FDCP1-LMTK3 was then assessed.

### Results

The results are shown in Table 2.

TABLE 2
IC₅₀ values for C28 derivatives

| | FDCP1- | FDCP1- |
|---|---|---|
| | parental IC₅₀ | LMTK3 IC₅₀ |
| Compound | (nM) | (uM) |
| 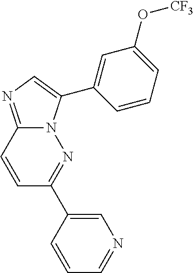 [SMILES: FC(F)(F)Oc1cccc(-c2cnc3ccc(-c4cccnc4)nn23)c1] | 7400 | 5500 |
| 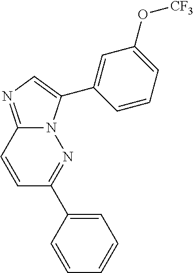 [SMILES: FC(F)(F)Oc1cccc(-c2cnc3ccc(-c4ccccc4)nn23)c1] | >10000 | 5800 |
| 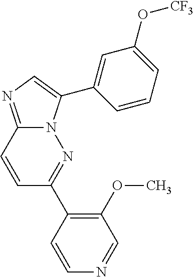 [SMILES: COc1cnccc1-c1ccc2ncc(-c3cccc(OC(F)(F)F)c3)n2n1] | >10000 | 8900 |
| 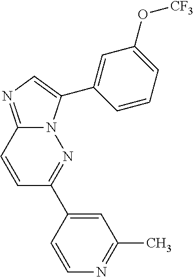 [SMILES: Cc1cc(-c2ccc3ncc(-c4cccc(OC(F)(F)F)c4)n3n2)ccn1] | 6800 | 5300 |
| 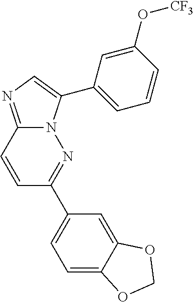 [SMILES: FC(F)(F)Oc1cccc(-c2cnc3ccc(-c4ccc5c(c4)OCO5)nn23)c1] | 9500 | 6500 |
| 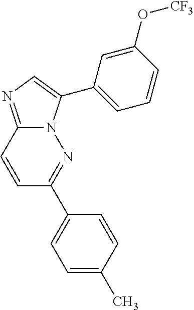 [SMILES: Cc1ccc(-c2ccc3ncc(-c4cccc(OC(F)(F)F)c4)n3n2)cc1] | >10000 | 8000 |
| 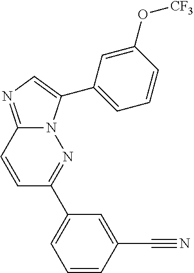 [SMILES: N#Cc1cccc(-c2ccc3ncc(-c4cccc(OC(F)(F)F)c4)n3n2)c1] | >10000 | 5500 |
| 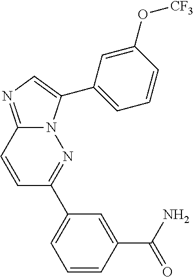 [SMILES: NC(=O)c1cccc(-c2ccc3ncc(-c4cccc(OC(F)(F)F)c4)n3n2)c1] | >10000 | 7700 |
| 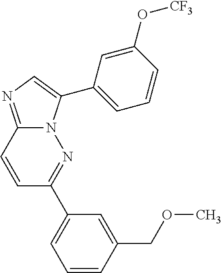 [SMILES: COCc1cccc(-c2ccc3ncc(-c4cccc(OC(F)(F)F)c4)n3n2)c1] | >10000 | 8900 |
| 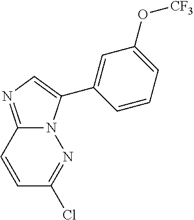 [SMILES: FC(F)(F)Oc1cccc(-c2cnc3ccc(Cl)nn23)c1] | >10000 | 9400 |

### CONCLUSIONS

Due to their knowledge of the structure of LMTK3, the inventors were able to adopt a high-throughput screening approach to identify compounds that could be promising candidates for drug development against LMTK3. Using this approach, the inventors were able to identify a compound, namely C28, which binds to and inhibits LMTK3 (ATP-competitive inhibitor) with high selectivity and demonstrates effective anticancer effects in a variety of cancer cell lines and in in vivo BC mouse models, apparently sparing the normal epithelium.

Interestingly, the inventors found that C28 promotes the proteasome-mediated degradation of LMTK3, which is of great importance considering the dual role of LMTK3 as a kinase and as a scaffold protein via which it can confer its biological effects. ATP competitive inhibitors of several different oncogenic protein kinases that depend on HSP90-CDC37 for their biological stability, have been shown to promote their degradation by antagonising binding of the CDC37 recruitment factor to the ATP-binding site of the kinase. This deprives the kinases of access to the chaperone system and channels them into ubiquitylation and subsequent degradation. As LMTK3 has the classic characteristics of an HSP90 ‘client protein’ it is likely that C28 is promoting its degradation through this ‘chaperone deprivation’ mechanism. The ability of C28 to abolish both the catalytic and the scaffolding functions of LMTK3, has allowed the inventors to identify both LMTK3 phospho-substrates as well as interacting partners that depend on LMTK3's scaffolding properties.

Mechanistically, C28 caused G2/M phase arrest and induction of apoptosis, a phenotype that is frequently observed following treatment with microtubule depolymerization agents. The inventors' experimental data revealed that C28 cannot bind directly to tubulin and affect its polymerization suggesting of an alternative mechanism of action. Interestingly, the inventors identified NUSAP1, a well-described MAP involved in bundling and stabilization of microtubules, to be down-regulated following treatment with C28 or after silencing of LMTK3. Combined, the inventors' findings propose that the pre-clinical therapeutic advantage of C28 stems from its effect on the LMTK3-targeted pathways linked to microtubule organization, acting differently from the established role of chemotherapeutic agents including vinca-alkaloids, taxanes or eribulin, which confer their cytotoxicity via their interactions with tubulin causing disruption of microtubule function. Moreover, C28 had no toxicity in normal tissues or the body weights in BC mouse models, while its anti-proliferative and pro-apoptotic effects on a non-transformed breast cell line (MCF12A) were significantly lower compared to other BC cell lines.

In aggregate, the development of oral LMTK3 inhibitors may have the potential for broad clinical utility, either as monotherapy or as a combinational therapy, (i.e. combined for example with aromatase inhibitors in ER⁺ BC in the same way the CDK4/6 inhibitors are). More precisely, in the case of TNBC despite immunotherapies being helpful at one level, there are no approved targeted therapies. Therefore, based on aberrant expression of LMTK3 in TNBC and work showing that genomic inhibition of LMTK3 leads to inhibition of cell proliferation, invasion and migration, an LMTK3 inhibitor would represent an attractive candidate for clinical trials. On the other hand, since the mechanism of endocrine and chemotherapy resistance in BC still remains largely un-explained, there remains a need to treat these patients in a more focused way, for example in the setting of progression on CDK4/6 inhibitors. Based on the results so far (in vitro, in vivo and clinical data from patient specimen cohorts), inhibition of LMTK3 may be important in tamoxifen (Tam) and doxorubicin (Dox) re-sensitization. Consequently, an LMTK3 inhibitor could be used alongside established therapies (e.g Tam, Dox) to increase the sensitivity of tumors and/or potentially overcome resistance.

### General Materials for the Above Examples

MG-132 (cat. no. 474790) and anisomycin from Streptomyces griseolus (cat. no. 176880-10MG) were purchased from Millipore and resuspended in DMSO (Millipore, cat. no. D/4125/PB08). Cyclohexamide (cat. no. 357420010) was purchased from Thermo Fisher Scientific and resuspended in DMSO (Millipore, cat. no. D/4125/PB08). Paclitaxel (cat. no. T1912-1MG) and colchicine (cat. no. C9754-100MG) were purchased from Sigma Aldrich and resuspended in DMSO (Millipore, cat. no. D/4125/PB08). ER-alpha (1:1000, cat. no. 8644), ubiquitin (1:1000, cat. no. 3936), phospho-HSP27 (Ser15) (1:1000, cat. no. 2404), phospho-HSP27 (Ser82) (1:1000, cat. no. 9709), HSP27 (1:1000, cat. no. 3936), phospho-Histone H3 (Ser10) (1:1000, cat. no. 3377), BCL2 (1:1000, cat. no. 2870), BCL-XL (1:1000, cat. no. 2764), cleaved-PARP (1:1000, cat. no. 5625), His-Tag (D3I1O) XP® (cat. no. 12698) antibodies as well as anti-rabbit IgG (1:5000, cat. no. 7074P2) and anti-mouse IgG (1:5000, cat. no. 7076P2) HRP-linked antibodies were purchased from Cell signaling. Phosphor-DYRK1/A (Tyr 321/273; 1:1000, cat. no. 12497) and phospho-TRKA (Tyr 680/681; 1:1000, cat. no. 11904) were purchased from SAB, FLAG antibody (1:1000, cat. no. F7425) was purchased from Sigma Aldrich, NUSAP1 (anti-ANKT) (1:1000, cat. no. STJ91598) was purchased from St John's Labs, GAPDH (1 μg/ml, cat. no. 39-8600) was purchased from Thermo Fisher Scientific, while TRKA (1:1000, cat. no. A01404), β-actin (0.1 μg/ml, cat. no. A00702-100) and a-tubulin (0.5 μg/ml or 1:100, cat. no. A01410-100) were purchased from GenScript. CLK2 (1:1000, cat. no. A7885), DYRKIA (1:1000, cat. no. A0595) and IRAK4 (1:1000, cat. no. A6208) were purchased from ABClonal. A Sigma Aldrich antibody (1:500, cat no. WH0114783M2) was used to detect total LMTK3, while an Abcam antibody (1:500, cat. no. 110516) was used to detect its kinase domain. pCMV6-LMTK3 overexpressing plasmid (RC223140) and the pCMV6 empty vector (PS100001) were purchased from Origene. All the other reagents, if not otherwise specified, were purchased from Thermo Fisher Scientific.

Cell Lines and Cloning

Parental FDCP1 cells were purchased from Advanced Cellular Dynamics, Inc. The FDCP1 interleukin 3 (IL³)-dependent cell line was grown in RPMI 1640 medium (Invitrogen) supplemented with 10% heat-inactivated FBS (Sigma Aldrich, cat. no. F7524-500ML), 1% L-Glutamine/Penicillin/Streptomycin (Sigma Aldrich, cat. no. G1146-100ML) and 10 ng/ml IL-3 (Genscript, cat. no. P01586).

To create a FDCP-LMTK3, FDCP1 cells were transduced with a pACD320 retroviral vector encoding the BCR protein fused to Flag epitope-tagged LMTK3 gene, which encompasses aa 134-444 (kinase domain). This fusion-donor approach is employed because different kinases often demonstrate preferential transformation capacity based on their ability to dimerize, which depends on the specific fusion partner deployed (Melnick J S, et al. (2006) An efficient rapid system for profiling the cellular activities of molecular libraries. Proc Natl Acad Sci USA 103(9)3153-3158) (fusions with other proteins including NPM, TPR, bTEL and nTEL did not result in high transactivation of LMTK3 as the one observed with BCR). All of the cells were incubated at 37° C. with 5% CO₂.

### General Synthetic Methods

General synthetic methods for producing various compounds of formula (I) are shown below in the form of retrosynthetic analysis.

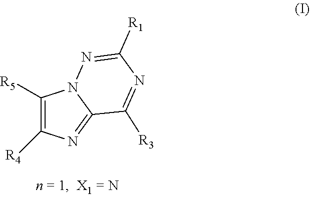

A retrosynthetic (working backwards) synthesis shows that we can start from an imidazole N-diazotise or N-nitrate and reduce, acetylation (to C, then ammonia-mediated cyclisation will furnish (1)

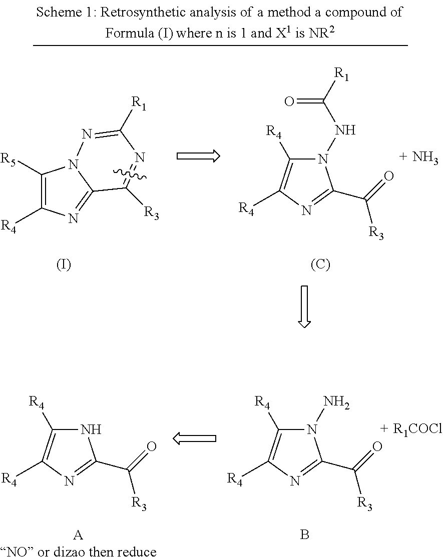

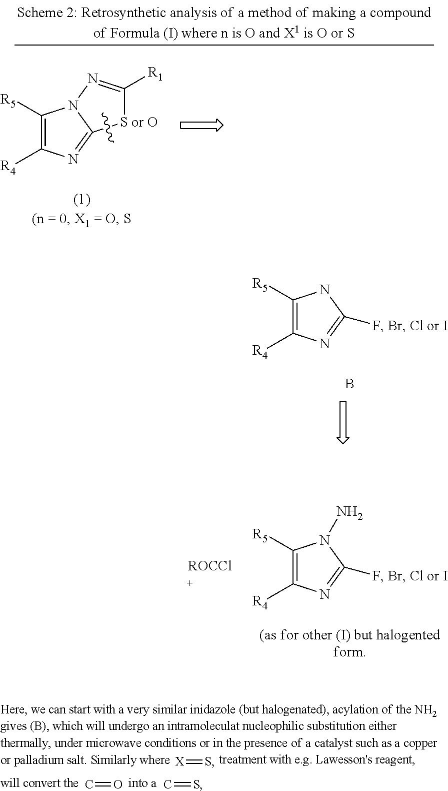

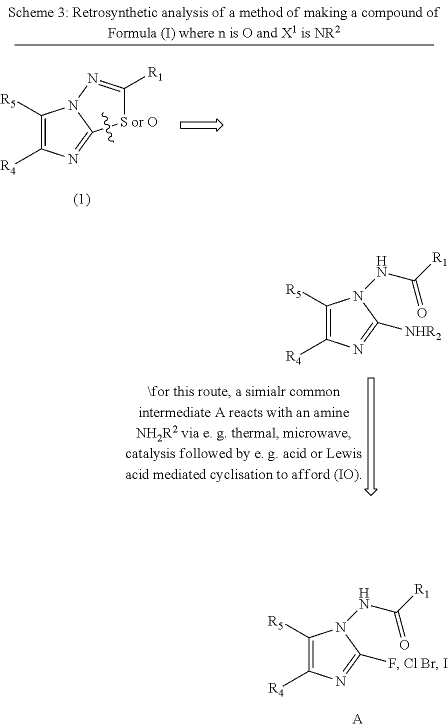

1. A method of treating, preventing or ameliorating a disease treatable by inhibiting Lemur tyrosine kinase 3 (LMTK3) in a subject, the method comprising administering to a subject in need of such treatment, a therapeutically effective amount of a compound of formula (1): 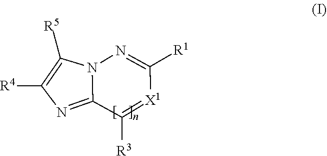
wherein R¹ is an optionally substituted C₆-C₁₂ aryl, an optionally substituted 5 to 10 membered heteroaryl, an optionally substituted 3 to 10 membered heterocycyl, L¹L²R⁸ or a halogen, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;
n is 0 and X¹ is S, O or NR²; or n is 1 and X¹ is CR² or N;
R² to R⁴ are independently hydrogen, a halogen, an optionally substituted C₁-C₁₅ alkyl, an optionally substituted C₂-C₁₅ alkenyl or an optionally substituted C₂-C₁₅ alkynyl;
R⁵ is an optionally substituted C₆-C₁₂ aryl, an optionally substituted 5 to 10 membered heteroaryl, an optionally substituted 3 to 10 membered heterocycyl, or L¹L²R⁸, wherein the aryl, heteroaryl or heterocycyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, OR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶; and
R⁶ and R⁷ are independently H, optionally substituted C₁-C₁₅ alkyl, optionally substituted C₂-C₁₅ alkenyl or optionally substituted C₂-C₁₅ alkynyl;
R⁸ is OR⁶, SR⁶, NR⁶R⁷, CONR⁶R⁷, CN, COOR⁶, NO₂, NR⁶COR⁷, NR⁶SO₂R⁷, OC(O)OR⁶, OC(O)NR⁶R⁷, OC(O)R⁶, an optionally substituted C₆-C₁₂ aryl, an optionally substituted 5 to 10 membered heteroaryl, an optionally substituted 3 to 10 membered heterocycyl, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;
L¹ is absent or is O, S or NR^{6;} and
L² is absent or is an optionally substituted C₁ to C₁₅ alkylene or an optionally substituted C₂ to C₁₅ alkylyne;
or a pharmaceutically acceptable complex, salt, solvate, tautomeric form or polymorphic form thereof.

2. (canceled)

3. The method according to claim 1, wherein the disease is cancer, attention deficit hyperactivity disorder (ADHD), hyper-sociability, a prepulse inhibition (PPI) deficit, cognitive dysfunction or a neurodegenerative disease.

4. The method according to claim 3, wherein the disease is cancer.

5. The method according to claim 1, wherein the compound is a compound of formula (II):

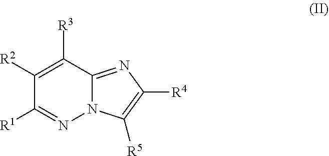

6. The method according to claim 5, wherein R² to R⁴ are each independently hydrogen, a halogen, an optionally substituted C₁-C₆ alkyl, an optionally substituted C₂-C₆ alkenyl or an optionally substituted C₂-C₆ alkynyl.

7. The method according to claim 6, wherein R² to R⁴ are each H.

8. The method according to claim 1, wherein R¹ is an optionally substituted C₆-C₁₂ aryl, an optionally substituted 5 to 10 membered heteroaryl, an optionally substituted 3 to 10 membered heterocycyl or a halogen.

9. The method according to claim 8, wherein R¹ is an optionally substituted phenyl, an optionally substituted thiophenyl, an optionally substituted thiazolyl, an optionally substituted tetrazolyl, an optionally substituted triazolyl, an optionally substituted pyridinyl, an optionally substituted pyridazinyl, an optionally substituted pyrimidinyl, an optionally substituted triazinyl, an optionally substituted 1,3-benzodioxolyl, an optionally substituted tetrahydropyranyl, an optionally substituted dihydropyranyl, an optionally substituted morpholinyl or chlorine.

10. The method according to claim 9, wherein R¹ is: 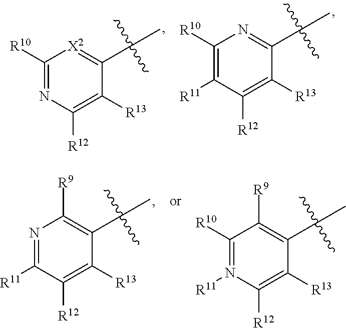
wherein X² is N or CR⁹; and
R⁹ to R¹³ are independently optionally substituted C₁-C₁₅ alkyl, optionally substituted C₂-C₁₅ alkenyl, optionally substituted C₂-C₁₅ alkynyl, halogen, OR⁶, NR⁶R⁷, CONR⁶R⁷, CN, COR⁶, COOR⁶, NO₂, NR⁶COR⁷, OC(O)OR⁶, OC(O)NR⁶R⁷ and OC(O)R⁶.

11. The method according to claim 10, wherein R¹ is Cl, phenyl,

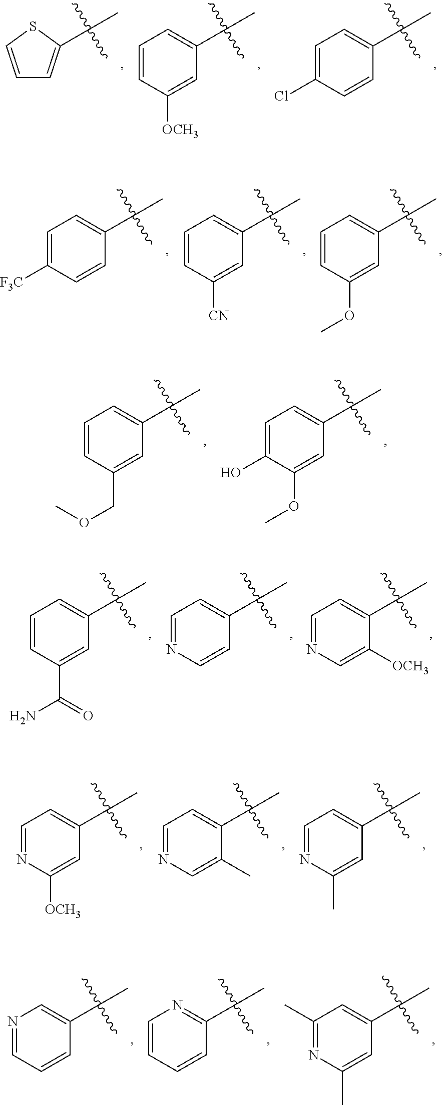

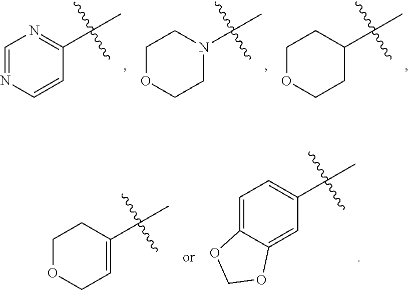 [SMILES: *c1ccncn1 ; *N1CCOCC1 ; *C1CCOCC1 ; *C1=CCOCC1 ; *c1ccc2c(c1)OCO2]

12. The method according to claim 11, wherein R¹ is

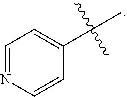 [SMILES: *c1ccncc1]

13. The method according to claim 5, wherein R⁵ is an optionally substituted phenyl or an optionally substituted 5 or 6 membered heteroaryl.

14. The method according to claim 13, wherein R⁵ is an optionally substituted phenyl or an optionally substituted 5 or 6 membered heteroaryl, wherein the phenyl or hereoraryl is optionally substituted with one or more substituents selected from the group consisting of optionally substituted C₁-C₆ alkyl, optionally substituted C₂-C₆ alkenyl, optionally substituted C₂-C₆ alkynyl, halogen, OR⁶, SR⁶, COR⁶ and CONR⁶R⁷, wherein R⁶ and R⁷ are H, optionally substituted C₁-C₆ alkyl, optionally substituted C₂-C₆ alkenyl or optionally substituted C₂-C₆ alkynyl.

15. The method according to claim 14, wherein R⁵ is 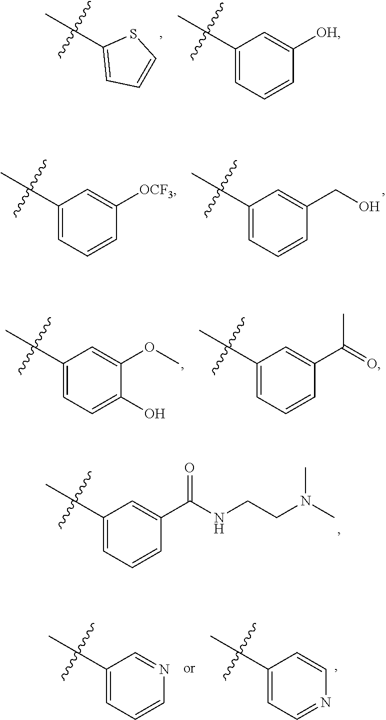
optionally wherein R⁵ is a phenyl substituted with OCF₃.

16. (canceled)

17. The method according to claim 1, wherein the compound of formula (I) is a compound of formula (100) to (122):

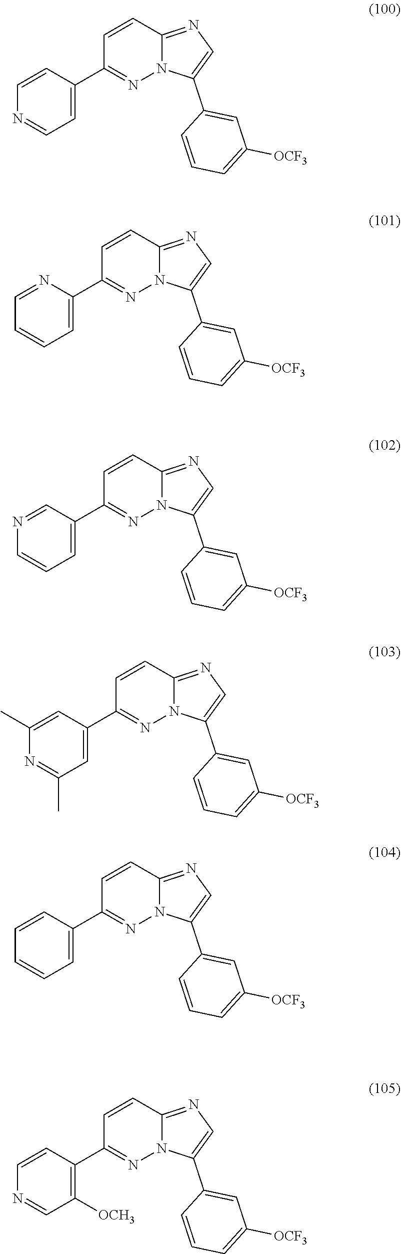 [SMILES: FC(F)(F)Oc1cccc(-c2cnc3ccc(-c4ccncc4)nn23)c1 ; FC(F)(F)Oc1cccc(-c2cnc3ccc(-c4ccccn4)nn23)c1 ; FC(F)(F)Oc1cccc(-c2cnc3ccc(-c4cccnc4)nn23)c1 ; Cc1cc(-c2ccc3ncc(-c4cccc(OC(F)(F)F)c4)n3n2)cc(C)n1 ; FC(F)(F)Oc1cccc(-c2cnc3ccc(-c4ccccc4)nn23)c1 ; COc1cnccc1-c1ccc2ncc(-c3cccc(OC(F)(F)F)c3)n2n1]

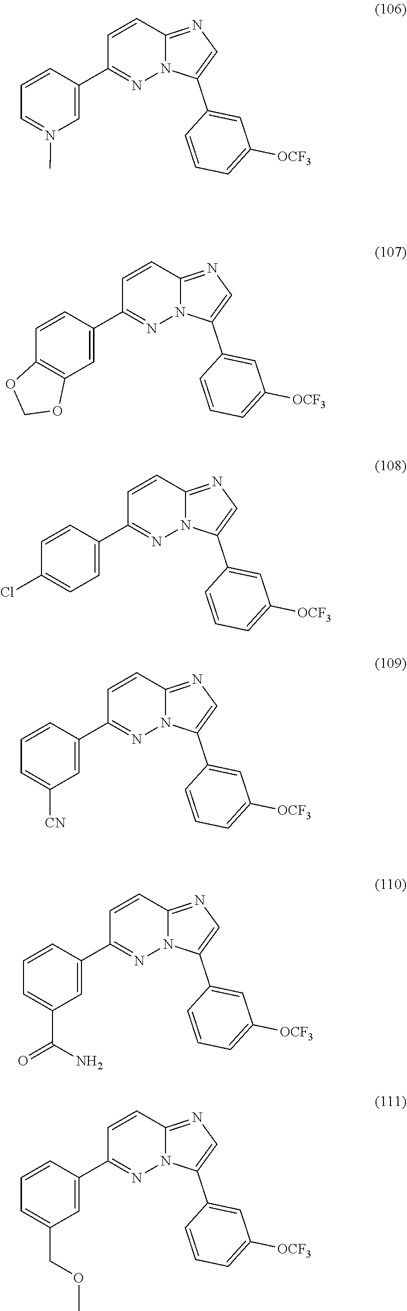

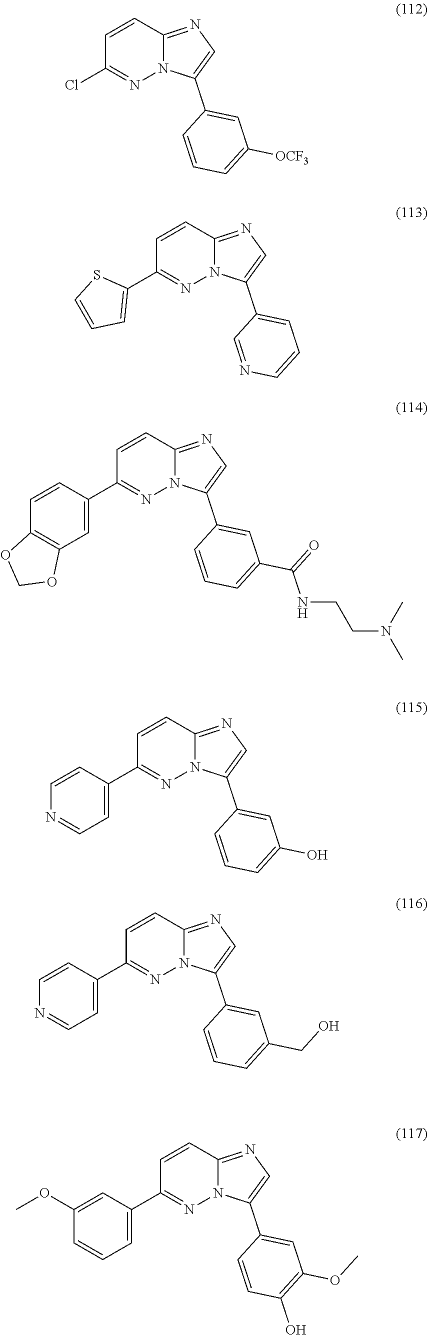

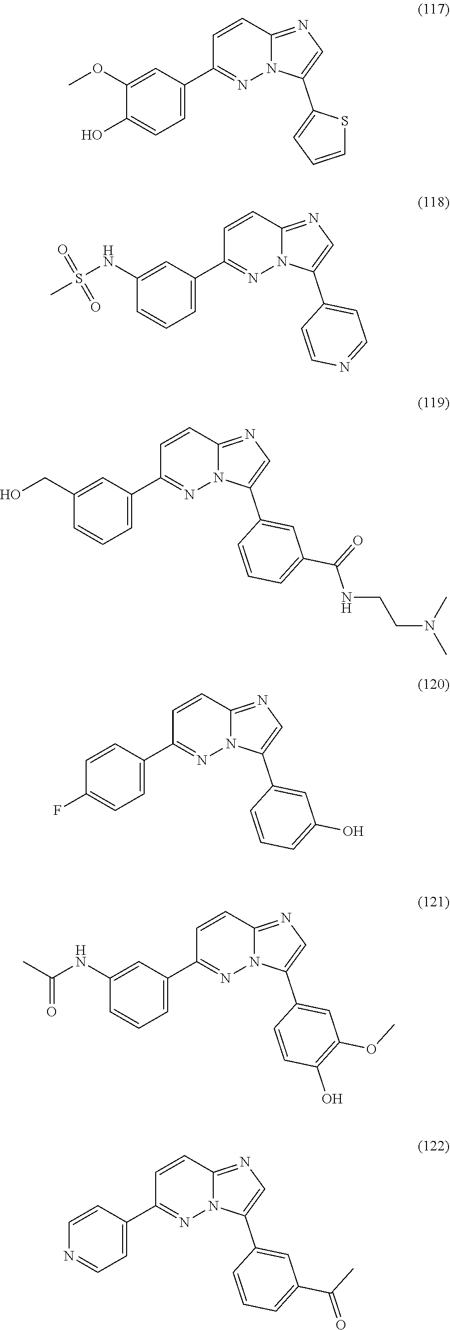

18. The method according to claim 1, wherein the compound is a compound of formula (III):

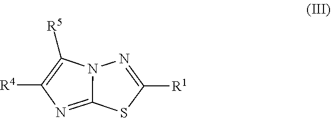

19. The method according to claim 18, wherein the compound is a compound of of formula (IIIa):

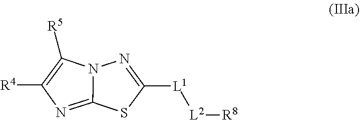

20. The method according to claim 18, wherein the compound is a compound of formula (200) to (204):

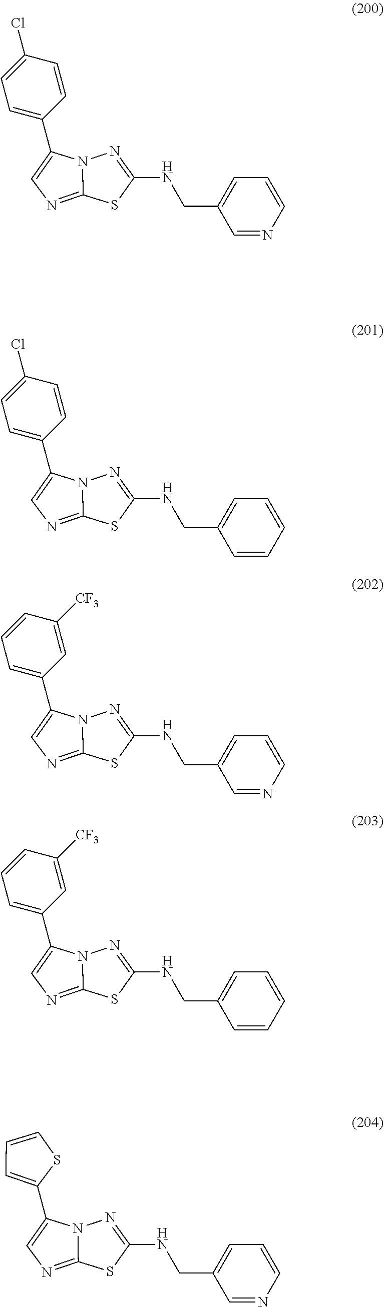 [SMILES: Clc1ccc(-c2cnc3sc(NCc4cccnc4)nn23)cc1 ; Clc1ccc(-c2cnc3sc(NCc4ccccc4)nn23)cc1 ; FC(F)(F)c1cccc(-c2cnc3sc(NCc4cccnc4)nn23)c1 ; FC(F)(F)c1cccc(-c2cnc3sc(NCc4ccccc4)nn23)c1 ; c1cncc(CNc2nn3c(-c4cccs4)cnc3s2)c1]

21. A pharmaceutical composition for treating cancer in a subject, the composition comprising a compound of formula (I) and a pharmaceutically acceptable vehicle, wherein the compound of formula (I) is: 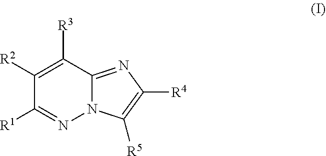
wherein R¹ is an optionally substituted C₆-C₁₂ aryl, an optionally substituted 5 to 10 membered heteroaryl, an optionally substituted 3 to 10 membered heterocycyl, L¹L²R⁸ or a halogen, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COOR⁶, NO₂, NR⁶COR⁷, OC(O)OR⁶, OC(O)NR⁶R⁷ and OC(O)R⁶;
R² to R⁴ are independently hydrogen, a halogen, an optionally substituted C₁-C₁₅ alkyl, an optionally substituted C₂-C₁₅ alkenyl or an optionally substituted C₂-C₁₅ alkynyl;
R⁵ is an optionally substituted C₆-C₁₂ aryl, an optionally substituted 5 to 10 membered heteroaryl, an optionally substituted 3 to 10 membered heterocycyl, or L¹L²R⁸, wherein the aryl, heteroaryl or heterocycyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, OR⁶, NR⁶R⁷, CONR⁶R⁷, CN, COOR⁶, NO₂, NR⁶COR⁷, OC(O)OR⁶, OC(O)NR⁶R⁷ and OC(O)R⁶; and
R⁶ and R⁷ are independently H, optionally substituted C₁-C₁₅ alkyl, optionally substituted C₂-C₁₅ alkenyl or optionally substituted C₂-C₁₅ alkynyl;
R⁸ is OR⁶, SR⁶, NR⁶R⁷, CONR⁶R⁷, CN, COOR⁶, NO₂, NR⁶COR⁷, OC(O)OR⁶, OC(O)NR⁶R⁷ and OC(O)R⁶;
L¹ is absent or is O, S or NR⁶;
L² is absent or is an optionally substituted C₁ to C₁₅ alkylene or an optionally substituted C₂ to C₁₅ alkylyne;
or a pharmaceutically acceptable complex, salt, solvate, tautomeric form or polymorphic form thereof.

22. A compound of formula (I): 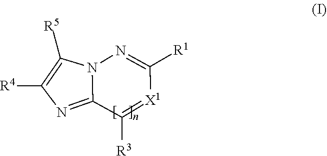
wherein R¹ is an optionally substituted. C₆-C₁₂ aryl, an optionally substituted 5 to 10 membered heteroaryl, an optionally substituted 3 to 10 membered heterocycyl, L¹L²R⁸ or a halogen, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;
n is O and X¹ is S, O or NR²; or n is 1 and X¹ is CR² or N;
R² to R⁴ are independently hydrogen, a halogen, an optionally substituted C₁-C₁₅ alkyl, an optionally substituted C₂-C₁₅ alkenyl or an optionally substituted C₂-C₁₅ alkynyl;
R⁵ is an optionally substituted C₆-C₁₂ aryl, an optionally substituted 5 to 10 membered heteroaryl, an optionally substituted 3 to 10 membered heterocycyl, or L¹L²R⁸, wherein the aryl, heteroaryl or heterocycyl is optionally substituted with one or more substituents selected from the group consisting of optionally substituted C₁-C₁₅ alkyl, optionally substituted C₂-C₁₅ alkenyl, optionally substituted C2-C₁₅ alkynyl, halogen, OR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶; and
R⁶ and R⁷ are independently H, optionally substituted C₁-C₁₅ alkyl, optionally substituted C₂-C₁₅ alkenyl or optionally substituted C₂-C₁₅ alkynyl;
R⁸ is OR⁶, SR⁶, NR⁶R⁷, CONR⁶R⁷, CN, COOR⁶, NO₂, NR⁶COR⁷, NR⁶SO₂R⁷, OC(O)OR⁶, OC(O)NR⁶R⁷, OC(O)R⁶, an optionally substituted C₆-C₁₂ aryl, an optionally substituted 5 to 10 membered heteroaryl, an optionally substituted 3 to 10 membered heterocycyl, wherein the aryl, heteroaryl or heterocyclyl is optionally substituted with one or more substituents selected from the group consisting of optionally substituted C₁-C₁₅ alkyl, optionally substituted C₂-C₁₅ alkenyl, optionally substituted C₂-C₁₅ alkynyl, halogen, O⁻, OR⁶, SR⁶, NR⁶R⁷, CONR⁶R⁷, CN, COR⁶, COOR⁶, NO₂, NR⁶COR⁷, NR⁶SO₂R⁷, OC(O)OR⁶, OC(O)NR⁶R⁷ and OC(O)R⁶;
L¹ is absent or is O, S or NR⁶; and
L² is absent or is an optionally substituted C₁ to C₁₅ alkylene or an optionally substituted C₂ to C₁₅ alkylyne;
or a pharmaceutically acceptable complex, salt, solvate, tautomeric form or polymorphic form thereof,
wherein compounds of formula (100), (113) to (122) and (200) are excluded: 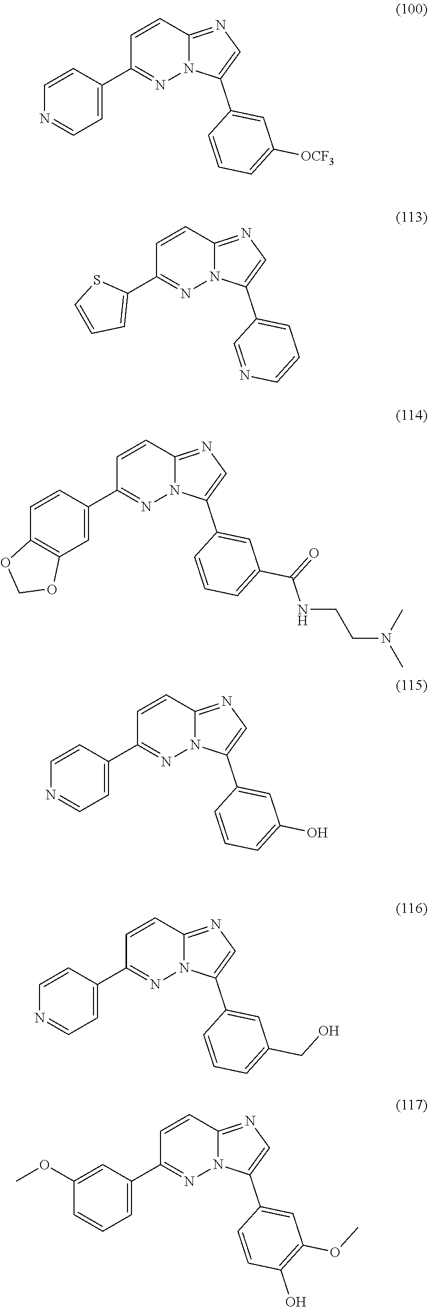 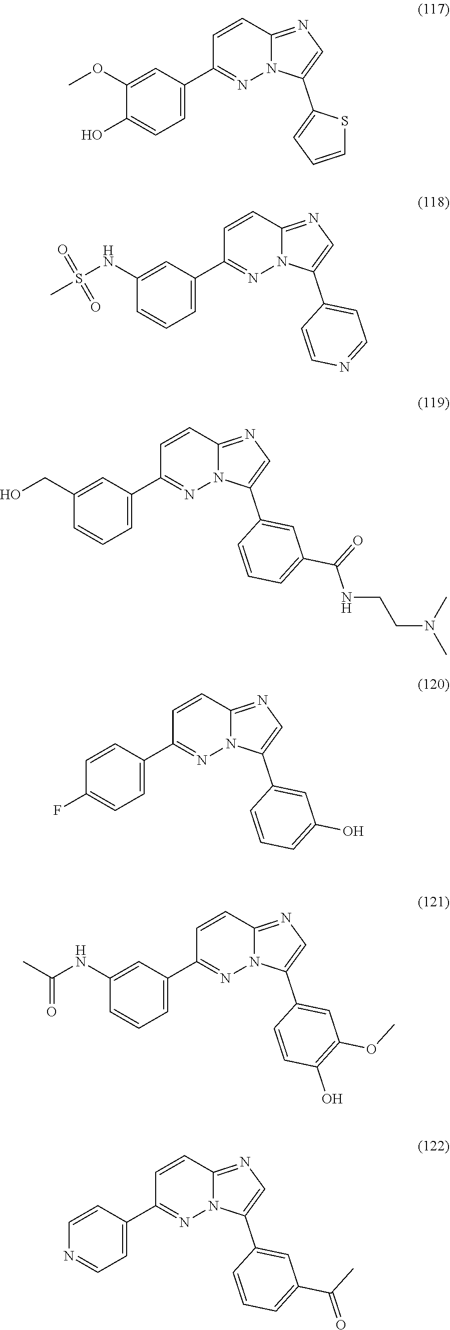 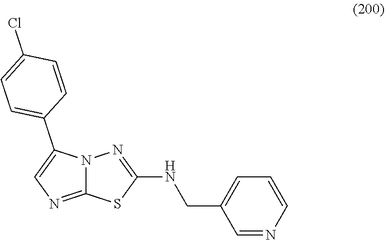 [SMILES: Clc1ccc(-c2cnc3sc(NCc4cccnc4)nn23)cc1]
